(19) United States
(12) Patent Application Publication
(10) Pub. No.: US 2007/0123776 A1
(43) Pub. Date: May 31, 2007

(54) Ultrasonic probing device with distributed sensing elements

(76) Inventors: Rami Aharoni, Rehovot (IL); Avram Matcovitch, Nesher (IL)

Correspondence Address: PEARL COHEN ZEDEK LATZER, LLP, 1500 BROADWAY 12TH FLOOR, NEW YORK, NY, 10036, US

(21) Appl. No.: 10/552,757

(22) PCT Filed: Feb. 23, 2004
(86) PCT No.: PCT/IL04/00172

§ 371 Date: Dec. 14, 2006

(30) Foreign Application Priority Data
Apr. 9, 2003 (IL) 155329

(51) Int. Cl. A61B 8/00

(52) U.S. Cl. 600/437

(57) ABSTRACT

A probing device for insertion into a duct to determine local parameters associated with the physical structure the duct at a selected region of the duct, and in particular variations in the physical structure along a predetermined length of interest. The probing device comprises: at least one of a plurality of waveguides incorporated in an elongated assembly designed to be inserted into the duct; at least one of a plurality of transmitters, spaced and distributed along a predetermined length of said at one of a plurality of waveguides incorporated in the elongated assembly, each capable of independently transmitting an acoustic signal of predetermined characteristics; a plurality of waveguides incorporated in the elongated assembly, each capable of receiving echoes of the acoustic signal, reflected off the structure of the duct; whe when each of said at least one of a plurality of transmitters generates an acoustic signal, echoes of the signal received by the plurality of receivers and received data associated with the echoes is processed by a processing unit to determine parameters of the physical structure at the region.

### FIELD OF THE INVENTION

The present invention relates to probes, especially ultrasonic probing devices that are operated or controlled using non-electrical transmission methods. More particularly, the present invention relates to an ultrasonic probing device with a miniature cross-section that is suitable for intravascular medical operations including diagnostics such as monitoring of coronal artery, or general vascular dimensions. The probing device is small enough to fit inside or be incorporated within a standard guidewire such as used in intervascular procedures.

### BACKGROUND OF THE INVENTION

Catheters having ultrasound capability at or adjacent to their tips that is affected by the transmittance of optical waves to and reception of optical signals from the tip of the catheter while converting the optical waves into ultrasonic waves and the ultrasound back into an optical signal are known in the art. An example of which is U.S. Pat. No. 5,944,687 “Opto-Acoustic Transducer for Medical Applications” disclosed by Benett et al. describing an optically activated transducer for generating acoustic vibrations in a biological medium. The transducer is located at the end of an optical fiber that may be located within a catheter. Energy for operating the transducer is provided optically by laser light transmitted through the optical fiber to the transducer. Pulsed laser light is absorbed in the working fluid of the transducer to generate thermal stress and consequent expansion of the transducer head such that it applies forces against the ambient medium. The transducer returns to its original state by a process of thermal cooling. Celliers et al. teaches in U.S. Pat. No. 6,022,309 “Opto-Acoustic Thrombolysis” a catheter-based device for generating an ultrasound excitation in biological tissue. Pulsed laser light is guided through an optical fiber to provide the energy for producing the acoustic vibrations. The optical energy is deposited in a water-based absorbing fluid, e.g. saline, thrombolytic agent, blood or thrombus, and generates an acoustic impulse in the fluid through thermoelastic and/or thermodynamic mechanisms. An additional patent disclosed by Sinofsky et al. named “Device for Use in Laser Angioplasty” discloses an apparatus for use in removing atherosclerotic plaque deposits in a blood vessel that comprises a high power laser, an elongated, flexible catheter adapted to be inserted into, and advanced through the blood vessel, a plurality of circumferentially arrayed optical fibers extending axially through the catheter, and an ultrasonic transducer at the distal end of the catheter for transmitting acoustical energy toward a selected area of the inner surface of a blood vessel in response to laser energy coupled through any one of the optical fibers and impinging upon the transducer. A detector proximal to the ultrasonic transducer is responsive to ultrasonic energy reflected from the blood vessel and produces a signal indicative of the tissue interfaces of the blood vessel. Laser energy can be transmitted from the high power laser through the same optical fiber used for the diagnostic procedure to ablate plaque in the blood vessel.

In a disclosure incorporated herein as a reference, PCT/IL02/00018 “Ultrasonic Transducer Probe”, Aharoni et. al. (not yet published) describe a compact cross-sectioned electromagneticl acoustic arrangement for generating and detecting ultrasound waves using an electromagnetic waveguide. The Acoustic generator comprises a source of electromagnetic radiation, a waveguide coupled to the source and at least one absorbing region defined in said waveguide, said region being selectively absorbing for portions of said radiation meeting at least one certain criterion and having significantly different absorbing characteristics for radiation not meeting said criterion, both of said radiation being suitable for conveyance through said waveguide, wherein said absorbing region converts said radiation into an ultrasonic acoustic field. Optionally, said region comprises a volumetric absorber. Alternatively or additionally, said region comprises plurality regions. The phenomenon of converting electromagnetic radiation to ultrasound is comprehensively described in PCT/IL02100018. It is emphasized that the devices described in the prior art differ from the acoustic generator described in PCT/IL02/00018 disclosure in at least one of the following aspects:
The prior art uses a fluid reservoir as the opto-acoustic conversion medium.
The prior art uses fluid positioned externally from the device as the opto-acoustic conversion medium.
The prior art uses angled metal targets as the opto-acoustic conversion medium.

In addition, prior art primarily relies on technologies that require a relatively large cross-section. Consequently, a central guide wire is used in order to guide the devices into the artery. Therefore, these designs necessarily require a significantly larger diameter than the guide wire itself. The ability to reduce the cross-section of the device, for example if it can be made to the guidewire itself, has many significant advantages for intravascular diagnostics and in particular for monitoring coronal artery dimensions as well as other medical applications.

### SUMMARY OF THE INVENTION

It is an object of the present invention to provide a new and unique ultrasonic transducer having a very small cross-section for diagnostics such as dimensional monitoring of an artery along its length.

It is another object of the present invention to provide a new and unique probe for diagnostics such as dimensional monitoring having a distributed array of sensing regions so as to monitor the variation of artery parameters over an extended artery length. The distributed array of sensing regions eliminates the need to mechanically relocate the device along in the artery in order to monitor artery cross-sectional parameters over a specified artery length.

It is thus provided in accordance with a preferred embodiment of the present invention a probing device for insertion into a duct having a physical structure to determine local parameters associated with the physical structure of the duct at a selected region of the duct, and in particular variations in the physical structure along a predetermined length of interest, the probing device comprising:
at least one of a plurality of waveguides incorporated in an elongated assembly designed to be inserted into the duct;
at least one of a plurality of transmitters, spaced and distributed along a predetermined length of said at least one of a plurality of waveguides incorporated in the elongated assembly, each capable of independently transmitting an acoustic signal of predetermined characteristics;
a plurality of receivers, spaced and distributed along a predetermined length of said at least one of a plurality of waveguides incorporated in the elongated assembly, each capable of receiving echoes of the acoustic signal, reflected off the structure of the duct;
whereby when each of said at least one of a plurality of transmitters generates an acoustic signal, echoes of the signal are received by the plurality of receivers and received data associated with the echoes is processed by a processing unit to determine parameters of the physical structure at the region.

Furthermore, in accordance with a preferred embodiment of the present invention, at least some of said at least one of a plurality of transmitters and said plurality of receivers are combined in the form of receiving and transmitting transducers.

Furthermore, in accordance with a preferred embodiment of the present invention, at least some of the transducers are piezo-electric transducers.

Furthermore, in accordance with a preferred embodiment of the present invention, each of said at least one of a plurality of transmitters, comprises an absorbing region within an optical fiber, the absorbing region made from material, which converts optical signals to acoustic signals. Furthermore, in accordance with a preferred embodiment of the present invention, each of said at least one of a plurality of transmitters, comprises at least one of a plurality of absorbing regions within an optical fiber, the absorbing regions made from material, which converts optical signals to acoustic signals.

Furthermore, in accordance with a preferred embodiment of the present invention, the absorbing regions are made of material that absorbs at different optical spectra, such that at least one of the absorbing regions are activated to generate acoustic signals at a certain optical spectrum, and other absorbing regions are activated to generate acoustic signals at one or more different optical spectra.

Furthermore, in accordance with a preferred embodiment of the present invention, the absorbing regions are made of material selected from the group containing: Copper-doped material with absorption spectrum at about 450 nm or shorter wavelengths, Alexandrite-doped material with absorption at about 850 nm or longer wavelengths, and Yitterbium-doped material with absorption in the range 1,000-1300 nm.

Furthermore, in accordance with a preferred embodiment of the present invention, each of said plurality of receivers comprises at least one of a plurality of optical fibers through which light can traverse and be modulated by the echoes.

Furthermore, in accordance with a preferred embodiment of the present invention, each one of said fibers, serving as a receiver, includes a reflecting element that effectively limits the extents of the fiber.

Furthermore, in accordance with a preferred embodiment of the present invention, the reflecting element comprises a Bragg grating optical element.

Furthermore, in accordance with a preferred embodiment of the present invention, at least some of said fibers serving as receivers are staggered along the length of interest to sensitize them to different regions along the length of interest.

Furthermore, in accordance with a preferred embodiment of the present invention, signals are processed by subtracting signals of two detecting fibers, such that the resulting signal corresponds to their region where the two fibers do not overlap.

Furthermore, in accordance with a preferred embodiment of the present invention, said fibers, serving as receivers, each include two reflecting elements and two types of light propagating in the fiber effectively forming two detection channels; the distal reflecting element serves to effectively limit the extent of the fiber for one of the detecting channels, and the proximal reflecting element serves to effectively limit the extent of the fiber for the other detecting channel; the differential signal between these two channels effects a sensitive region local to the separation of the two reflecting elements.

Furthermore, in accordance with a preferred embodiment of the present invention, at least some of said sensitive local regions are staggered along the length of interest to sensitize them to different regions along the length of interest.

Furthermore, in accordance with a preferred embodiment of the present invention, received signals are processed to account for different phases in each receiver in conjunction with a knowledge of physical separation between the receivers so as to effect a circumferential resolution in the device.

Furthermore, in accordance with a preferred embodiment of the present invention, at least one of the two reflecting elements comprises a Bragg grating optical element, and the two channels are differentiated by wavelength.

Furthermore, in accordance with a preferred embodiment of the present invention, at least one of the two reflecting elements comprises a polarization-dependent reflector, and the two channels are differentiated by polarization.

Furthermore, in accordance with a preferred embodiment of the present invention, at least one of the two reflecting elements comprises a spatially selective element, reflecting one or more guided modes, and the two channels are differentiated by guided modes.

Furthermore, in accordance with a preferred embodiment of the present invention, each of said plurality of receivers comprises at least one of a plurality of optical fibers through which light can traverse and be modulated by the echoes and which incorporates several wavelength-dependent reflectors, such that each effectively limits extent of a certain optical wavelength traveling in the fiber; the position of at least some of these reflecting elements is distributed along the length of the interest, sensitizing each wavelength to a different positions along the length of interest.

Furthermore, in accordance with a preferred embodiment of the present invention, the received signals are processed to account for the different phases in each receiver in conjunction with a knowledge of physical separation between the receivers so as to effect a circumferential resolution in the device.

Furthermore, in accordance with a preferred embodiment of the present invention, each of said at least one of a plurality of transmitters, comprises at least one absorbing region within a multicore optical fiber, the absorbing region made from material, which converts optical signals to acoustic signals, and wherein at least one of the cores serve as at least one receiver.

Furthermore, in accordance with a preferred embodiment of the present invention, some of the cores serving to generate the acoustic signals have larger lateral cross section than those serving for detection.

Furthermore, in accordance with a preferred embodiment of the present invention, the cores in the said multicore optical fiber, serving as receivers, include a reflecting element that effectively limits the extent of each of the receiver cores and sensitizes each on to a different positions along the length of interest.

Furthermore, in accordance with a preferred embodiment of the present invention, the reflecting element comprises a Bragg grating optical element.

Furthermore, in accordance with a preferred embodiment of the present invention, said cores, serving as receivers, include two reflecting elements and two types of light propagating in each core effectively forming two detection channels; the distal reflecting element serves to effectively limit the extents of the core for one of the detecting channel, and the proximal reflecting element serves to effectively limit the extent of the core for the other channel; the differential signal between these two channels effects a sensitive region local to the separation of the two reflecting elements.

Furthermore, in accordance with a preferred embodiment of the present invention, at least one of the two reflecting elements comprises a Bragg grating optical element, and the two channels are differentiated by wavelength.

Furthermore, in accordance with a preferred embodiment of the present invention, at least one of the two reflecting elements comprises a polarization-dependent reflector, and the two channels are differentiated by polarization.

Furthermore, in accordance with a preferred embodiment of the present invention, at least one of the two reflecting elements comprises a spatially selective element, selectively reflecting one or more guided modes, but not reflecting other such modes, and the two channels are differentiated by different guided modes.

Furthermore, in accordance with a preferred embodiment of the present invention, said predetermined length of the elongated structure extends to approximately 30 mm.

Furthermore, in accordance with a preferred embodiment of the present invention, there is provided a probing device for insertion into a duct having a physical structure to determine local parameters associated with the physical structure of the duct at a selected region of the duct, and in particular variations in the physical structure along a predetermined length of interest, the probing device comprising:
an elongated assembly designed to be inserted into the duct;
a plurality of transmitters, spaced and distributed along a predetermined length of said elongated assembly, each capable of independently transmitting an acoustic signal of predetermined characteristics;
at least one of a plurality of receivers, spaced and distributed along a predetermined length of said elongated assembly, each capable of receiving echoes of the acoustic signal, reflected off the structure of the duct;
whereby when each of said plurality of transmitters generates an acoustic signal, echoes of the signal are received by the at least one of a plurality of receivers and received data associated with the echoes is processed by a processing unit to determine parameters of the physical structure at the region.

Furthermore, in accordance with a preferred embodiment of the present invention, said plurality of transmitters and said at least one of a plurality of receivers are combined in the form of receiving and transmitting transducers.

Furthermore, in accordance with a preferred embodiment of the present invention, at least some of the transducers are piezo-electric transducers.

Furthermore, in accordance with a preferred embodiment of the present invention, each of said plurality of transmitters, comprises an absorbing region within an optical fiber, the absorbing region made from material, which converts optical signals to acoustic signals.

Furthermore, in accordance with a preferred embodiment of the present invention, each of said plurality of transmitters, comprises at least one of a plurality of absorbing regions within an optical fiber, the absorbing regions made from material, which converts optical signals to acoustic signals.

Furthermore, in accordance with a preferred embodiment of the present invention, the absorbing regions are made of material that absorbs at different optical spectra, such that at least one of the absorbing regions are activated to generate acoustic signals at a certain optical spectrum, and other absorbing regions are activated to generate acoustic signals at one or more different optical spectra.

Furthermore, in accordance with a preferred embodiment of the present invention, the absorbing regions are made of material selected from the group containing: Copper-doped material with absorption spectrum at about 450 nm or shorter wavelengths, Alexandrite-doped material with absorption at about 850 nm or longer wavelengths, and Yitterbium-doped material with absorption in the range 1,000-1300 nm.

Furthermore, in accordance with a preferred embodiment of the present invention, each of said at least one of a plurality of receivers comprises at least one of a plurality of optical fibers through which light can traverse and be modulated by the echoes.

Furthermore, in accordance with a preferred embodiment of the present invention, said fibers, serving as receivers, each include two reflecting elements and two types of light propagating in the fiber effectively forming two detection channels; the distal reflecting element serves to effectively limit the extent of the fiber for one of the detecting channels, and the proximal reflecting element serves to effectively limit the extent of the fiber for the other detecting channel; the differential signal between these two channels effects a sensitive region local to the separation of the two reflecting elements.

Furthermore, in accordance with a preferred embodiment of the present invention, each of said fibers serving as receivers are staggered along the length of interest to sensitize them to different regions along the device.

Furthermore, in accordance with a preferred embodiment of the present invention, the received signals are processed to account for the different phases in each receiver in conjunction with a knowledge of their physical separation so as to effect a circumferential resolution in the device.

Furthermore, in accordance with a preferred embodiment of the present invention, at least one of the two reflecting elements comprises a Bragg grating optical element, and the two channels are differentiated by wavelength.

Furthermore, in accordance with a preferred embodiment of the present invention, at least one of the two reflecting elements comprises a polarization-dependent reflector, and the two channels are differentiated by polarization.

Furthermore, in accordance with a preferred embodiment of the present invention, at least one of the two reflecting elements comprises a spatially selective element, selectively reflecting one or more guided modes, and the two channels are differentiated by guided modes.

Furthermore, in accordance with a preferred embodiment of the present invention, each one of said fibers, serving as a receiver, includes a reflecting element that effectively limits the extents of the fiber.

Furthermore, in accordance with a preferred embodiment of the present invention, the reflecting element comprises a Bragg grating optical element.

Furthermore, in accordance with a preferred embodiment of the present invention, each of said fibers serving as receivers are staggered in their length to sensitize them to different regions along the length of interest.

Furthermore, in accordance with a preferred embodiment of the present invention, signals are processed by subtracting signals of two adjacent detecting fibers, such that the resulting signal corresponds to their region where the two fibers do not overlap.

Furthermore, in accordance with a preferred embodiment of the present invention, each of said plurality of receivers comprises an optical fiber through which light can traverse and be modulated by the echoes and which incorporates several wavelength-dependent reflectors, such that each effectively limits extent of a certain optical wavelength traveling in the fiber; the position of these reflecting elements is distributed along the predetermined length of the device, sensitizing each wavelength to a different positions along the assembly.

Furthermore, in accordance with a preferred embodiment of the present invention, the received signals are processed to account for the different phases in each receiver in conjunction with a knowledge of their physical separation so as to effect a circumferential resolution in the device.

Furthermore, in accordance with a preferred embodiment of the present invention, each of said at least one of a plurality of transmitters, comprises at least one absorbing region within a multicore optical fiber, the absorbing region made from material, which converts optical signals to acoustic signals, and wherein several of the cores serve as one or more receivers.

Furthermore, in accordance with a preferred embodiment of the present invention, some of the cores serving to generate the acoustic signals have larger lateral cross section than those serving for detection.

Furthermore, in accordance with a preferred embodiment of the present invention, the cores in the said multicore optical fiber, serving as receivers, include a reflecting element that effectively limits the extent of each of the receiver cores and sensitizes each on to a different positions along the assembly.

Furthermore, in accordance with a preferred embodiment of the present invention, the reflecting element comprises a Bragg grating optical element.

Furthermore, in accordance with a preferred embodiment of the present invention, said fiber, serving as receiver, includes two reflecting elements and two types of light propagating in the fiber effectively forming two detection channels; the distal reflecting element serves to effectively limit the extents of the fiber for one of the detecting channel, and the proximal reflecting element serves to effectively limit the extent of the fiber for the other channel; the differential signal between these two channels effects a sensitive region local to the separation of the two reflecting elements.

Furthermore, in accordance with a preferred embodiment of the present invention, at least one of the two reflecting elements comprises a Bragg grating optical element, and the two channels are differentiated by wavelength.

Furthermore, in accordance with a preferred embodiment of the present invention, at least one of the two reflecting elements comprises a polarization-dependent reflector, and the two channels are differentiated by polarization.

Furthermore, in accordance with a preferred embodiment of the present invention, at least one of the two reflecting elements comprises a spatially selective element, reflecting one or more guided modes, and the two channels are differentiated by guided modes.

Furthermore, in accordance with a preferred embodiment of the present invention, said predetermined length of the elongated structure extends to approximately 30 mm.

Furthermore, in accordance with a preferred embodiment of the present invention, there is provided a system for determining local parameters associated with a physical structure of a duct at a selected region of the duct, and in particular their variation of a predetermined length of interest, the system comprising:
at least one of a plurality of waveguiding structures incorporated with an elongated assembly designed to be inserted into the duct;
a plurality of transmitters, spaced and distributed along a predetermined length of said at least one of the plurality of waveguides incorporated with the elongated assembly, each capable of transmitting an acoustic signal of predetermined characteristics;
at least one of a plurality of receivers, spaced and distributed along a predetermined length of said at least one of the plurality of waveguides incorporated with the elongated assembly, each capable of receiving echoes of the acoustic signal, reflected off the structure of the duct;
a processing unit for processing the received echoes;
a controller for actuating and controlling the operation of the generator and the processing unit,
   whereby when each of said at least one of a plurality of transmitters generates an acoustic signal, echoes of the signal are received by at least one of the plurality of receivers and received data associated with the echoes is processed by a processing unit to determine parameters of the physical structure at the region.

Furthermore, in accordance with a preferred embodiment of the present invention, there is provided a method for determining local parameters associated with a physical structure of a duct at a selected region of the duct, and in particular variations in the physical structure along a predetermined length of interest, the method comprising:
providing a system comprising:
a probing device comprising at least one of a plurality of waveguiding structures incorporated within an elongated assembly designed to be inserted into the duct; at least one of a plurality of transmitters, spaced and distributed along a predetermined length of said at least one of the plurality of waveguiding structures incorporated with the elongated assembly, each capable of transmitting an acoustic signal of predetermined characteristics; and at least one of a plurality of receivers, spaced and distributed along a predetermined length of said at least one of the plurality of waveguides incorporated with the elongated structure, each capable of receiving echoes of the acoustic signal, reflected off the structure of the duct;
a processing unit for processing the received echoes;
a controller for actuating and controlling the operation of the generator and the processing unit,
inserting the probing device within the duct at a desired target;
generating an acoustic signal by each of said at least one of a plurality of transmitters;
receiving echoes of the acoustic signal;

processing data associated with the echoes by the processing unit to determine parameters associated with a physical structure of a duct at the desired region.

### BRIEF DESCRIPTION OF THE FIGURES

In order to better understand the present invention, and appreciate its practical applications, the following Figures are provided and referenced hereafter. It should be noted that the Figures are given as examples only and in no way limit the scope of the invention as defined in the appending Claims. Like components are denoted by like reference numerals.

FIG. 1 illustrates a sectional side view of a duct with an ultrasonic probing device having distributed sensing elements in accordance with a preferred embodiment of the present invention placed within an artery with a confinement in its' cross-section.

FIGS. 2a-2d illustrate a preferred embodiment of the present invention using a bundle of separate optical fibers each capable of transmitting and receiving ultrasound. FIG. 2a shows the staggering of the fiber tips to affect a distributed transducer as is the purpose of the current invention, FIG. 2b shows a tightly packed cross-section of the fiber bundle for 7 elements; FIG. 2c shows a tightly packed cross-section of the fiber bundle for 10 elements; and FIG. 2d shows a tightly packed cross-section of the bundle for 14 elements.

FIG. 3a illustrates a longitudinal sectional view of an ultrasonic probing device in accordance with a preferred embodiment of the present invention having a set of absorbing regions and a plurality of waveguiding structures within one assembly.

FIG. 3b illustrates an axial cross-sectional view of the probing device shown in FIG. 2A with equal diameter cores each transmitting and detecting ultrasound

FIG. 3c illustrates an axial cross-sectional view of the probing device shown in FIG. 2A with one large core for transmission of ultrasound, and a set of smaller diameter cores for detecting ultrasound.

FIGS. 4a-4f illustrate the signal forms resulting from the build-up in signal cross-talk for different sensing elements in the probe of the present invention.

FIGS. 5a and 5b illustrate cross-sectional views of a probing device in accordance with a preferred embodiment of the present invention centered in the artery lumen (FIG. 5a), or eccentric with respect to the artery lumen (FIG. 5b).

FIG. 6 illustrates a an expanded view of the longitudinal section of a probing device showing the additional concept of dual-channeled detection cores which are implemented with the aid of a partial reflector located between the last absorbing generator to which that core is sensitive, and the penultimate generating absorber in the device.

FIG. 7 illustrates a sectional side-view of a probing device in accordance with another preferred embodiment of the present invention having a multi-waveguiding structure provided with staggered reflectors.

FIG. 8 illustrates a side view of a probe in accordance with yet another preferred embodiment of the present invention having a multi-waveguiding structure where different wavelengths are used to address different sensors along the detecting waveguides.

FIG. 9 illustrates a side-view of a probe in accordance with yet another preferred embodiment of the present invention having a set of wavelength discerning absorbers for multiplexing the generation of ultrasound along different absorbing regions.

FIGS. 10a and 10b illustrate a schematic representation of the spectral absorption curves for materials suitable for use in wavelength division transmitting core as a part of a preferred embodiment for the present invention. FIG. 10a shows separate band-pass absorption spectra, and FIG. 10b shows overlapping spectra, which are acceptable as long as distinct portions at each wavelength do not overlap.

FIG. 11 illustrates an optical ultrasonic probing system in accordance with a preferred embodiment of the present invention.

FIG. 12 illustrates the probing device of the present invention in an arrangement for guiding surgical tools along an assembly containing the device.

FIG. 13 illustrates a sectional view of a proximal connector in accordance with another preferred embodiment of the present invention, with dynamically aligning means for its separate cores.

FIG. 14 illustrates a proximal connector for a probing device of the present invention with self-aligning features for its separate cores.

FIG. 15 illustrates a proximal connection arrangement where the probing device of the present invention is connected to the system by a set of standard connectors, and in addition the sensor is cut at a distance from the connectors where it is mounted in a lateral self-aligning mount. This mount can rotate the transducer parts relatively to each other for accurate registration of the inner cores.

FIG. 16 is a schematic illustration of pulling a multi-core fiber to meet the requirements of the present inventions.

FIG. 17 a schematic illustration of manufacturing a multi-core fiber to meet the requirements of the present inventions.

### DETAILED DESCRIPTION OF THE PRESENT INVENTION AND THE FIGURES

Confinements in the cross-section of arteries occur as a result of a variety of artery illnesses, such as plaque or thrombosis. This situation often requires intravascular intervention. Typically, the surgeon must locate and measure the confined regions in order to properly position a device for expanding the artery, such as inflating balloons or by a device for applying artery-wall support such as a stent. The ability to measure and monitor the dimensions of the artery in preparation for, during, and/or after the procedure of balloon inflation or stent positioning presents a significant advantage for the surgeon. In accordance with a preferred embodiment of the present invention, a probe is provided for diagnostic operations such as monitoring the dimensions of the artery; a feature of this probe relates to its relatively small cross-section. In fact, the cross-section is small enough so as to allow the entire probe to fit inside a standard guidewire (whose typical diameter is in the order of 1 French, i.e. about 0.34 mm) that is used to guide surgery tools through arteries. Adding sensing features to the guidewire significantly improves its performance and applicability, combining its conventional mechanical function: guiding different surgical tools to a selected location within the artery, with sensing and diagnostic capabilities: locating the malignant region, mapping the extent of the malignant artery, and, for example, measuring the external and internal artery diameters. More advanced diagnostic operations, such as accurate imaging of the artery cross-section, or even characterization of the malignant tissue, can be implemented with a similar concept using a greater number of sensing elements and suitable signal analysis procedures.

State of the art dedicated diagnostic tools must be inserted along the guidewire to the required position to provide various sensing functions. Such diagnostic tools must be removed before surgical tools are inserted. A guidewire with sensing features in accordance with the present invention alleviates the necessity for the entire procedure of inserting and removing the diagnostic tools. Moreover, by using the device of the present invention, the surgeon can monitor the artery simultaneously with any treatment operations. These capabilities provides for a real-time process control tool that need not be removed prior to the insertion of any surgical tool.

In practice the lumen measurements that are most significant to the surgeon relate to the minimal, maximal, and average lumen diameter, and the artery wall-thickness at every artery section. Such statistical values are sufficient for most practical purposes. Optionally, additional angular details, such as the variation of the distance to the internal wall around the circumference of the artery's cross-section, may also be important. As the amount of data collected by the probe at every artery section increases, it can provide more information on the geometry of the artery's cross-section; essentially if the full potential of the data generated by the present invention is exploited, a high-resolution imaging probe can be implemented. The basic diameter/wall-thickness measurements are sufficient for identifying a malignant area when the measurements are distributed over a sufficient length of the artery. Typically, a malignant region in the artery is limited to several millimeters in length; it is very seldom that narrowed regions extend to an inch (25 mm). Consequently, a sensor with the capability of detecting the variation of the artery dimensions over a distance exceeding one inch (25 mm), preferably on the order of thirty millimeters, is capable of identifying the non-uniform and non-monotonic change in a malignant artery profile.

The required distributed sensing capability can be achieved, in principle, by moving a single sensor within a device such as a catheter over the required artery length. This method is referred to as “pull-back”, where a catheter incorporating a sensor near its distal tip is drawn back by pulling the proximal end over the distance of interest. While it is possible to use the “pull-back” procedure in connection with a probe of the present invention, such a procedure would compromise some of the inherent advantages of the present invention. The device of the present invention combines mechanical guiding and diagnostic functions. The “pull-back” procedure requires the surgeon to re-insert the device through the malignant region after each measurement. In the preferred embodiments of the present invention, as is comprehensively discussed herein, an array of sensing regions distributed over the length of the probe serve to monitor artery parameters along a predetermined length without the need for any scanning motion.

The underlying concept of the present invention relates to a series of local sensors, each comprising transmitting means and receiving means, distributed along a predetermined length, and designed to acquire information pertinent to the cross-section of the artery at the location of each sensor. Although each of these sensors performs a simple, elementary measurement, the overall data acquired using this arrangement is sufficient to provide significant information on the artery structure and its variations along a predetermined length. Only a basic performance is required of each transducer. Therefore the transducers can be physically very small, rendering the device of the present invention suitable for insertion, interrogation and mapping of minute bodily ducts.

Notwithstanding the basic concept of minimal transducer capabilities to minimize the size of the probing device of the current invention, the probing device can encompass broader application capabilities. As described in the following, phase information gathered from different sensors can be used to enhance the lateral as well as the circumferential resolution of the basic device. Furthermore, the detected ultrasound carries information related to the composition, texture and other material properties of the artery walls. Therefore, in addition to extracting the profile of the artery, the present probing device is also capable of characterizing its material properties; this is an invaluable capability for differentiating between malignant and healthy regions within the duct.

A variety of different technologies may be used to Implement the individual sensors in the distributed arrangement of the present invention, including, but not limited to, piezoelectric devices, acousto-magnetic devices, EMAT (electromagnetic acoustic transducers), and acousto-optic devices. Nevertheless, as mentioned in the following, in cases where the sensor is to be disposable, opto-acoustic methods, relying on optical fiber technologies, have a favorable advantage.

An ultrasonic probing device having distributed sensors, in accordance with a preferred embodiment of the present invention, is based on electromagnetic waveguides that transmit radiation to generate ultrasonic signals from distributed ultrasonic transducers. For convenience only, and without loss of generality, we refer in the following to optical waveguides, specifically optical fibers, and light as an example of such waveguides and such radiation. Nevertheless the meaning of these expressions is maintained in its broader sense whereby “light” should be taken here to represent any form of electromagnetic radiation, and “optical fiber”, or “fiber”, any form of electromagnetic waveguide. The echoes of these ultrasonic signals are reflected off the walls of the artery and a portion of them is redirected back onto the probe. These signals, in turn, modulate designated portions of the light traveling in the probe. By demodulating the reflecting light of the probe as it exits on return from the sensing region of the device, it is possible to detect the ultrasonic echoes and diagnose various parameters of the artery, including, for example, the dimension of the artery lumen and its wall-thickness. Although it is conceptually straight-forward to implement such a device with piezo-electric transducers, such implementation requires the insertion of wires through the device. The fundamental advantage of the proposed acousto-optic arrangement is the absence of any such wiring, potentially simplifying the construction and minimizing its manufacturing costs in high-volume-production. The production cost is most significant when a disposable device is desired, such as is common with similar medical invasive devices.

There are different ways to implement the distributed probing device of the present invention. In general, the preferred embodiments can be classified into three major arrangements (and combinations thereof): a) a distributed array of sensors, each capable of transmitting and receiving ultrasonic signals independently of the rest of the sensors; b) one transmitting arrangement that transmits ultrasonic signals and a plurality of receiving sensors; and c) a plurality of independent transmitters generating ultrasonic waves and one receiver that detect received signals. The approach of configuration (a) is advantageous as it generates a set of independent ultrasonic signals that can be analyzed in one recursive procedure. Nevertheless, as the compactness of the sensor is of primary importance here, the use of one of the other two arrangements, which necessarily require less physical sensors, may be more suitable in many cases. Naturally any combination of the above classes can also be useful, such as a small number of transmitters in connection with a larger number of receivers and any permutation of such an arrangement.

Fundamentally, a transmitter is constructed within the probe of the present invention, by providing one or more absorbing regions within an optical waveguide. Light pulses transmitted through a fiber can cause the absorbing region to generate ultrasound signals. The basic thermo-elastic effect exploited in the probe and various transmitting configurations have been comprehensively described in PCT/IL02/00018 (not yet published). The following succinct description is repeated here for completeness. In the present invention, the description is limited to the phenomenon of thermo-elastic generation of ultrasound: light incident on an absorbing region heats it abruptly. Provided the heating is significantly faster than the thermal dissipation processes (conduction and radiation), a condition that can readily be met in practice, thermal stresses are generated. The thermal stresses propagate as acoustic waves. Absorbing regions that absorb the incident radiation of different geometries and embodiments are possible. There can be one absorbing region, or a plurality of absorbing regions that are dense enough to absorb all the intensity of the incident radiation such that no portion of the absorbed wavelength is transmitted past the absorbing region. Alternatively, a portion of the energy at the absorbed wavelength is transmitted through the region, while another portion is absorbed. Another alternative is that multiple absorbing regions can be provided for a specific wavelength, each region absorbs a portion of the light and transmits a portion of the light. The absorbency of the regions may be designed to provide a uniform (or patterned) thermal distribution so as to generate a specific form of ultrasonic field.

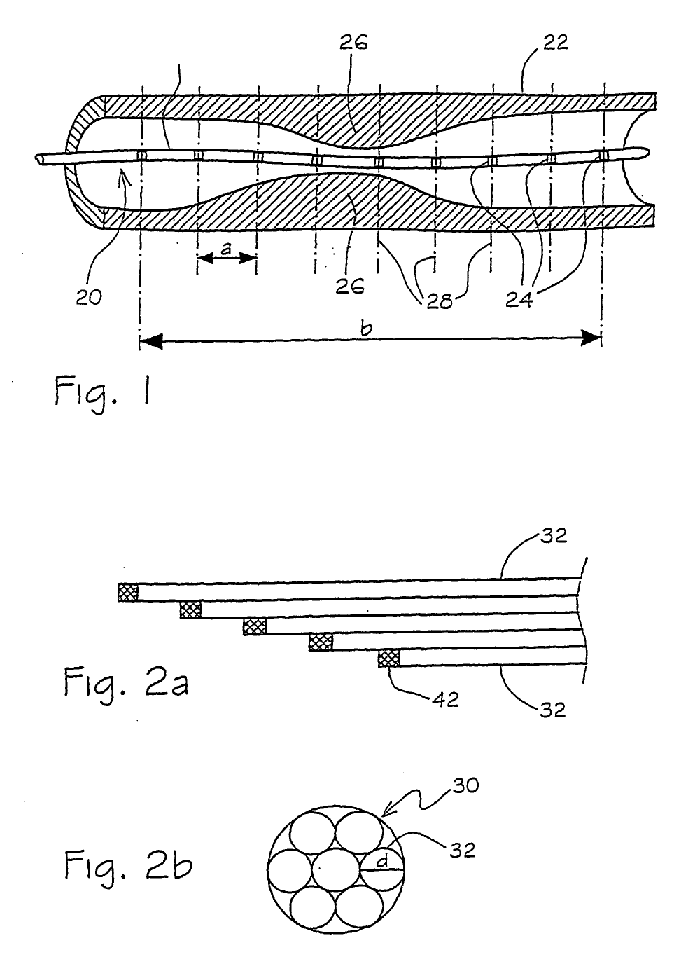

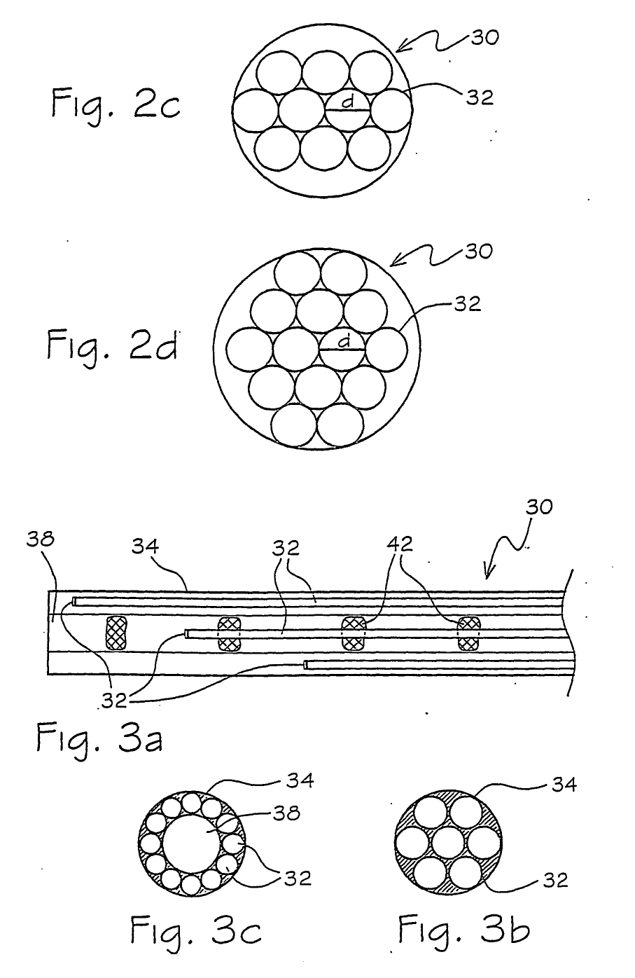

Reference is now made to FIG. 1 illustrating a sectional side-view of a bidily duct, such as an artery 22 containing an ultrasonic probing device 20 having distributed sensing regions 24 in accordance with a preferred embodiment of the present invention. The profile of a duct 22 is significantly narrowed due to an illness, such as plaque 26 or trombosis. An ultrasonic probing device 20 having several, distributed sensors 24 (FIG. 1 is given for brevity only, illustrating the basic concept of the invention. For details of preferred embodiments see FIGS. 2-3, 7-9,) is placed within duct 22. Each sensor is capable of extracting data on the local cross-section as indicated schematically by the vertical dash-dot lines 28 in FIG. 1. The sensors, 24 are spaced apart, extending over and beyond the full length of the narrowing 26. As mentioned above, the extent of malignant regions in arteries is usually limited. The ability to monitor statistical parameters of the duct at independent points along the duct, over a sufficient length, can serve to identify malignant regions where the internal duct geometry exhibits a non-monotonic variation as compared to adjacent regions. Typically, malignant regions extend no more than 13 mm (half an inch), and very seldom reach a length of 25 mm (1 inch). Therefore, if the effective sensing length of the probe is at least 25 mm, or preferably about 30 mm (denoted “b” in the FIG. 1), the probe is well capable of detecting such malignant regions. The spacing “a” between the sensors 24 determines the longitudinal resolution of the probe: the effective longitudinal resolution of the probe increases as this spacing decreases. For most practical purposes, a separation in the order of 3-5 mm is sufficient to effectively monitor the narrowing, so that an array of six to ten sensing elements in a probe would usually be sufficient. Obviously these numbers are illustrative only and the principle of the present invention can be expanded to include longer detection lengths, shorter, or irregular spacing between the absorbers. As noted above, different technologies can be used to implement the sensors in the probe of the present invention. Without loss of generality, the following describes an acousto-optical configuration. Piezo-electric components, for example, can serve the same purpose.

The desired effect of a distributed electromagnetic radiation-based ultrasonic probing device can be implemented in a variety of configurations. In general one can provide a set of independent sensing and detecting elements each capable of either generating ultrasound signals or detecting at a specified position, or alternatively capable of both functions. Such an exemplary embodiment is shown in FIGS. 2a and 2b for an array of transmitting/receiving optical fibers, each in accordance with the disclosure of PCT/IL02/00018. Each such fiber includes one or more regions, often close to its distal end, which are capable of exciting ultrasound by absorbing radiation transmitted towards it through the fiber; the same fiber is also capable of detecting the reflected ultrasound by optical means. The fiber-ends are staggered (FIG. 2a) so as to form a distributed array of sensors as described above. The packing of the fibers is shown schematically in FIG. 2b, for the case of 7 elements, in FIG. 2c for 10 elements and in FIG. 2d for 14 elements of diameter d. Evidently the overall diameter of the assembly is 3d, 4d and 4.5d, respectively. To maintain the entire assembly to within 240 μm, thereby enabling the assembly to fit in a standard guide wire of external diameter of 1 French˜340 μm, the individual elements should be limited in diameter to 240/3=80 μm, 240/4=60 μm, and 240/4.5=53 μm, respectively. Fibers of these diameters are common. Such an implementation has significant advantages on the system level whereby the same basic sensor/detector element is used repeatedly. The main drawbacks of this implementation relate to the limited power that can be used to drive the transducers, and to the discontinuities in the detecting sensor, which will scatter the ultrasound traversing through the assembly's lateral cross-section. The power in such a waveguide is limited both by the ability to couple the power into the waveguide and the power damage thresholds on the couple-in and couple-out surfaces. These are power density limitations and using larger waveguide cross-sections allows the transmission of greater power to generate stronger ultrasound signals. The loss of signal, both on the transmission and the detection due to ultrasound scattering from the fiber boundaries further reduces the available signal. Other drawbacks of this implementation include the complexity of the assembly process that would inhibit low-cost mass production procedures, the need for multiple high-power sources, or a means for switching them between the different fibers and a complex mechanical interconnection to the proximal end of the device that is, on the one hand small enough to fit in the surgical devices that are to be guided over it into position, and still transfer the optical signals with minimal loss for all the fibers, on the other hand.

An alternative preferred embodiment comprises a monolithic probing device assembly as illustrated in FIGS. 3a through 3b. Referring first to FIG. 3b, showing an axial cross-section of the device 30, this embodiment incorporates several cores 32 in a single cladding 34; this approach alleviates the losses due to ultrasound scatter within the assembly's cross-section as the cores and cladding offer similar acoustic properties and essentially form a continuum through which the ultrasound is transmitted without appreciable effects. We note that we refer to cores here as a means of guiding the light traversing along the device in a confined cross-section. Such guiding cores can be implemented by variation in the local refractive index in a variety of profiles (step-wise, gradual, W- or M-shaped etc.) or is can be implemented with hollow in the fiber. In principle this arrangement can utilize the same characteristics of the multi-fiber approach above, fibers being replaced with cores, and each core responsible for transmission and detection of ultrasound at a different location. A different approach, depicted in FIG. 3c, uses a larger core 38 for transmission of excitory radiation and consequent ultrasound generation from absorbing regions distributed along its length 42, and an array of smaller cores 32 for detection. Here the transmission is multiplexed as described below. As the number of distribute sensors grows, the transmission in this arrangement is increasingly more effective than for the separate-fiber arrangement of FIG. 2 or the equal-core arrangement of FIG. 3b for two reasons: a) the effective cross-sectional area that can be provided for the transmission radiation is larger, and b) the effective power density limit for the transmission radiation, which is expected to partly travel in the cladding and adjacent cores, can be increased. Relating to a typical overall assembly diameter of 240 μm as above, we can envisage a central core 38 of 210 μm diameter. Assuming 10 transmitters in series, and a power density (Pd) limit of Pd/π, one can deliver Pd×210²/10=4,410 Pd to each transmitter here as compared to Pd×60²=3,600 Pd in the separate waveguide implementation. Similar ratio's are found for the 7 element and 14 element situations: Pd×210²/7=6,300 Pd, and Pd×210²/14=3,150 Pd for the central transmitter case, as compared to Pd×80²=6,400 Pd and Pd×53²=2,809 Pd, respectively. Cleary for few elements the independent probing device arrangement is beneficial but as the number of elements increases the single element offers a better power performance. For the larger number of elements the 20% power delivery improvement is further enhanced by practical considerations of the larger numerical aperture of the monolithic approach. On the other hand, the peak power density delivered by separate cores to their absorbers is higher. Nevertheless, if wavelength division multiplexing is use, there is no need to distribute the available power between the absorbing regions and the single, large generation core is preferrable. For reference below we also indicate that the additional cores, reserved for detection, are, in this configuration on the order of 5-10 μm.

Referring now to FIG. 3a illustrating a longitudinal sectional view of an ultrasonic probing device 30 in accordance with a preferred embodiment of the present invention having a set of absorbing regions 42 and a plurality of individual guiding cores 32, or more generally, waveguiding structures, within a single assembly. The multi-core waveguide 30 comprises wave-guiding cores 32 of varying properties and positions across its lateral section. The central core 38 is adapted to transfer an electromagnetic pulse, a train of pulses, or any other waveform that is suitable for generating the required ultrasonic field. The propagated radiation of the electromagnetic pulse can be infrared, ultraviolet or visible light. A plurality of absorbers 42 is provided along body 30, wherein absorbers 42 are adapted to generate ultrasound waves. The generation of the ultrasound waves occurs immediately after an absorber absorbs radiation propagating in the central core 38, heats up as a result and expands abruptly emitting an ultrasonic wave. The timing of the generation from each absorber is controlled by the timing of the transmission of the irradiation pulses. In certain applications a simultaneous generation from all absorbers is required. Alternatively the absorbers are activated in a predetermined sequence. The absorption of each absorber is predetermined by selecting the right material according to its absorbing and resonance properties, so that the relative power dissipated in each absorber complies with a predetermined pattern. As an example, the power between the absorbers can be uniformly distributed so that similar ultrasonic intensities are generated from each absorber. If for any reason the absorbers are not similar, or a non-uniform set of ultrasonic waveforms is desired, other power proportions between the absorbers can be used.

When an electromagnetic pulse is transmitted through core 38 while the ultrasonic probing device is in a duct (as shown in FIG. 1), the propagated ultrasound waves generated by absorbers 42 are transmitted to the surrounding medium within the duct and to the duct walls. Portions of the waves that are reflected from the inner surface of the duct wall are referred to as front-wall (FW) echoes. In a similar way, additional ultrasonic reflections occur at other acoustic impedance discontinuities such as the intermediate layers within the artery wall, or the outer wall of the duct that is referred to as the back-wall (BW). These echoes propagate back through the medium of the duct towards waveguide 30. A portion of the echoes is transmitted into the fiber, where they interact with the plurality of cores 32, the cores adapted to guide electro-magnetic radiation (infra-red, ultraviolet, or visible light) towards the distal end of the device.

Cores 32 are elongated and pass along waveguide 30. In order to better understand the positioning of detecting cores 32, reference is now made to FIG. 3c illustrating a radial cross-sectional view (in plane A-A) of the probe shown in FIG. 3a. Peripheral cores 32 are distributed about the central core 38, however, as shown in FIG. 3a, these cores may have different effective lengths. Each peripheral core 32 extends beyond a different absorber 42 so that each peripheral core is adapted to receive a different portion of the echoes that are being reflected from the walls and medium of the artery. Radiation is transmitted through peripheral cores 32 from the proximal side (not shown in the Figures) of probing device 30 and is reflected back from the waveguides distal end by a reflecting optical element 44. The reflections propagate towards the proximal end of the probe to be processed by a signal-processing unit (see further explanation hereinafter). When no ultrasound is present, this counter-propagating radiation serves to establish a reference state. Ultrasound signal that traverses cores 32 disturbs and modulates the counter-propagating radiation. The disturbance effect can be detected and demodulated at the distal end of the probe to replicate the form and timing of the ultrasonic signal.

The distribution of lengths of cores 32 (as described in FIG. 3a) is established in order to differentiate between echoes generated by different absorbers, as explained hereinafter. Each guiding core receives signals that originated from a different set of absorbers. For example, the longest core 46 extends beyond the most distal absorber and traverses all of absorbers 42 available in probing device 30. Therefore, core 46 detects signals ensuing from all the absorbers and echoes reflected back towards the regions surrounding the absorbers. On the other hand, core 48 extends only beyond the most proximal absorber and in fact terminates before reaching regions of other absorbers. Therefore, core 48 is not affected by signal generated or reflected to the regions of other absorbers other than the most proximal absorber. In the same manner, each subsequent core is affected only by absorbing regions it traverses through. If one focuses on core 48, it detects disturbances generated by all the absorbing reagions proximal to its reflecting end (to the right of the end of core 48). The subsequent core, 46, detects disturbances from all the regions that affect core 48, and in addition from an absorbing region distal to the refecting end of core 48. Therefore, subtracting from the signal detected by core 46 the signal detected by core 48 renders the signal of the subsequent absorber. As the detecting cores are arranged to end in-between absorbing regions, there are always two cores for which the difference in their detectd signal corresponds to one absorbing region. In this manner, the signals of each of the absorbers can be demultiplexed, each detected signal from a specific core is subtracted from that of the preceding cores.

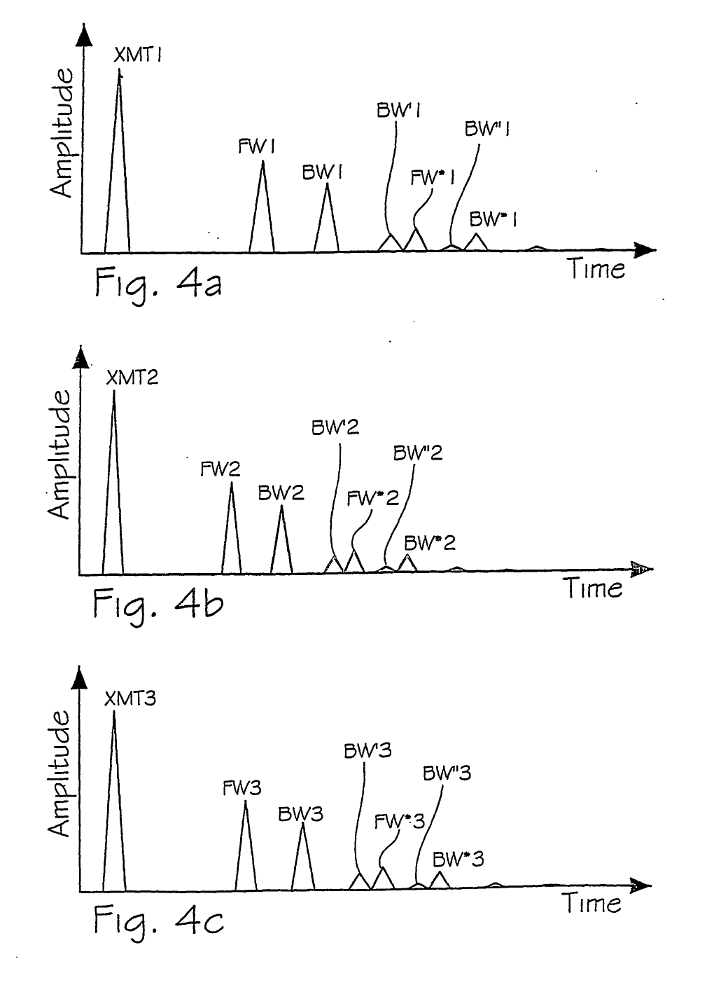

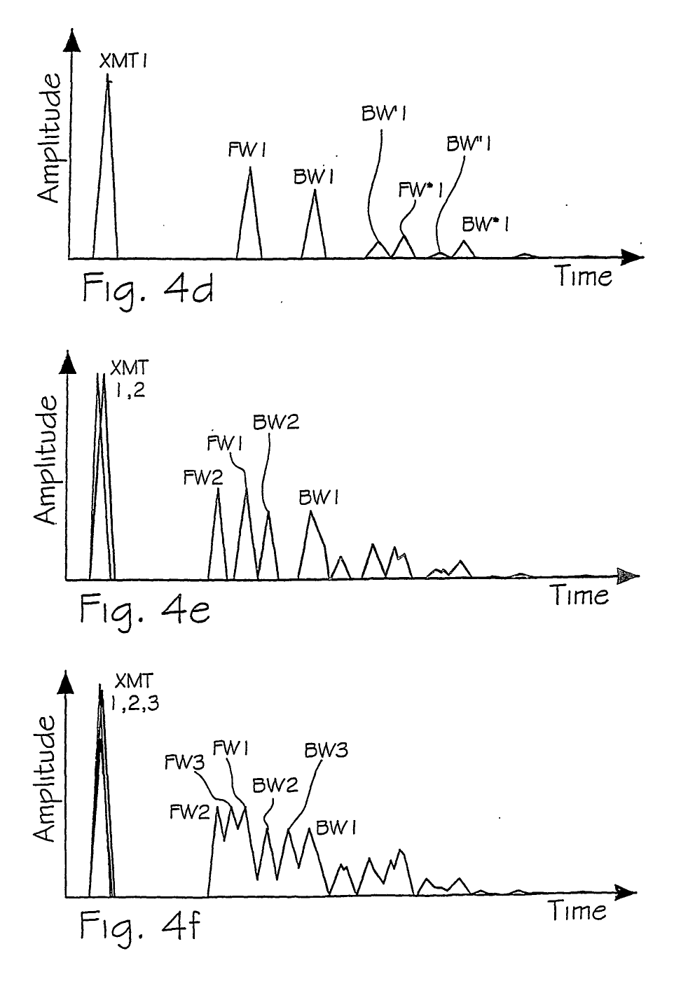

Reference is now made to FIG. 4 depicting a schematic illustration of a signal formed as a result of the build-up in signal cross-talk for different cores in the probe of the present invention. An example for the demultiplexing method is schematically described for a three-core/three-absorber arrangement. FIGS. 4a through 4c show the anticipated signals received by three independent detectors setup to detect the transmission signals from each one of the absorbing regions independently. The regions are numbered from 1 to 3. In these the transmitted (XMT) signal component is detected first immediately after it traverses the distance between the absorbing region and the detecting sensor. Next to arrive at the detector are, in sequence, the front-wall (FW) echo and the back-wall (BW) echo which are reflected off the front (inner face) and back (outer face) of the ducts wall. In addition to these main signals one finds several secondary signal sets, for example, multiple reverberations inside the wall thickness (marked BW′, BW″, etc) and multiple reverberations between the device's surface and the duct wall (marked FW*, BW* etc.). While the transmission signals occur in this example nearly simultaneously, the timing of the reflected signals for each sensor depends on its relative position within the probing device assembly and its relative distance to the duct wall. These distances are, in general, different for each sensor, but the duct wall thickness is necessarily the same. In this way, the time lapsed to the FW in each case differs, but the timing between the FW and the BW are similar. FIGS. 4d, 4e and 4f show the actual signal expected when the sensing is effected by wave-guiding cores that are staggered along the probing device so that the core extending up to region 3 (FIG. 4f) also passes by, and is therefore sensitive to, signals transmitted by regions 2 and 1. The resulting waveform detected by this core is a superposition of all three signals ensuing from the three transmitting regions. Similarly, the core that extends up to region 2 (FIG. 4e) also passes near region 1 and therefore it is sensitive to both the first and the second signals and detects a superposition of the two. In the proposed scheme, the signals of the core reaching region 3 (FIG. 4f) is demultiplexed by subtracting the signals from region 2 and 1 (FIG. 4e). Similarly the signals from the core reaching region 2 are demultiplexed by subtracting the detected signal from region 1 (FIG. 4d). In this manner each signal is demultiplexed by performing only one subtraction operation; the signal of the (N−1)^{th} core needs be subtracted from that of the N^{th} core, to obtain the net signal for the N^{th} transmitter. In the following account the subtracted signal is referred to as the reference signal, and the core which detects it is referred to as the reference core. The signal for N=1 is not multiplexed, and it requires no reference core.

It is noted, however, that for a good subtraction performance, the different timing of the signals due to the different locations of each detector need to be accounted for. Assuming the detecting cores are evenly distributed around the transmitting core, the direct XMT signal is expected to arrive in all cores simultaneously. Nevertheless the timing difference between the detection in one core and its reference core is directly related to the effective point of reflection from the artery (see below) and relative distance from this point to each core. To estimate the severity of these effects for typical core geometries we return to the example values of FIG. 3b: a central core of 210 μm, and, say 10 detecting cores of 5 μm diameter equally spaced on a diameter of 225 μm. For these dimensions the maximal separation between two adjacent cores is on the order of 2×π×112 μm/10˜70 μm, which for glass material correspond to a timing error of 12 ns; and plastic material 24 ns. Such timing errors are less significant as the frequency of operation is reduced, comprising only a 12% of the period of 10 MHz but 36% of a 30 MHz signal. Situations where these timing errors do not introduce a detrimental effect and measures to compensate for such timing errors are discussed in the following. Also considered are methods to turn these timing errors into an advantage for improving the information content that can be derived from the signals of the probe.

The description above refers to a one-dimensional model for the ultrasonic echoes. This model applies to the special case where the artery lumen is a perfect circle and the probe is positioned coaxially to the lumen. In this case the one-dimensional model holds as all the reflections from the circumference of the artery wall arrive at the same time to the probe, and the result is the waveforms shown in FIGS. 4a, 4b and 4c. In practice, the artery's symmetry is imperfect and there is no assurance that the probe is centered with respect with the artery's lumen. A more realistic model takes into account the eccentricity of the probe position in the lumen. In such an eccentric geometry, the probe still transmits the ultrasound uniformly in a radial direction, but due to off-radial reflections from the artery walls, only small portions of the artery wall reflect. The primary reflections are along the diameter passing through the location of the probe; short path and long path reflections are expected. In principle, a similar effect occurs for non-circular lumen whereby small portions of the circumference reflect. There may be several such reflecting points, but unless the probe is very close to the center of the lumen, each absorber will generate a separate set of signals. The relative delay of these signals relates to the eccentricity of the probe's location and the geometry of the lumen. In principle, collecting the relative delay data in each channel can serve to buildup a details pertaining to the true geometry of the lumen. In any case, it is relatively straightforward to derive the statistical parameters of the lumen, such as the minimal diameter, the maximal diameter and the average diameter.

Returning to FIG. 3a, cores 32 are distributed along probing device 30. This distribution necessarily adds a secondary effect to the description hereinabove in terms of the actual phase with which the signals arrive to the peripheral cores. The small perturbation in the location of the peripheral waveguides introduces a phase shift that can be used to enhance the data from which the profile of the duct is reconstructed if the signals incident on the fiber originate from the same source. As noted herein above, one expects two regimes: when the lumen is circular and the sensor is centered, essentially all reflections coincide at the same timing on the peripheral waveguides; and when either the probe is off-centered, or the lumen is elliptical (or otherwise non-circular), the reflections are confined to small portions of the lumen circumference, necessarily arriving at different timings to the peripheral waveguides.

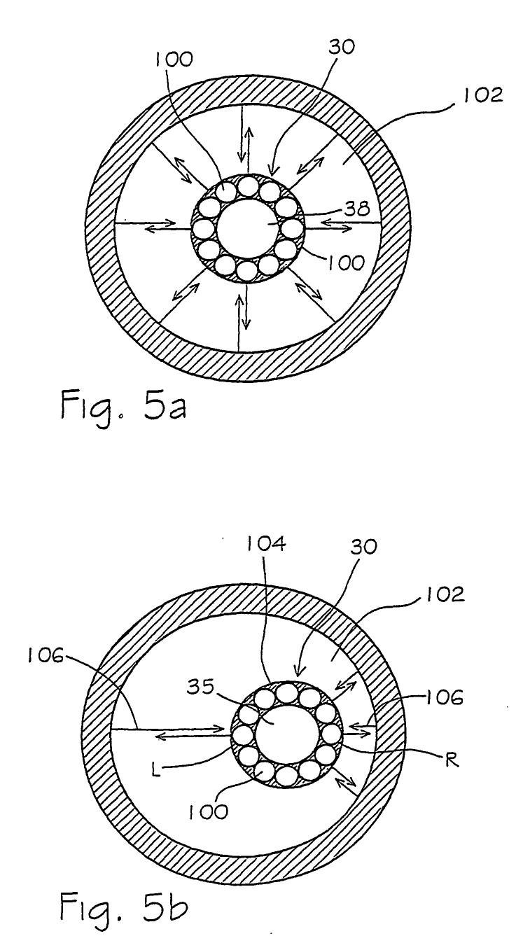

Reference is now made to FIG. 5a and 5b that illustrate cross-sectional views of a probing device in accordance with a preferred embodiment of the present invention placed concentric (in FIG. 5a) and eccentric (in FIG. 5b) to the lumen, respectively. In the former case (FIG. 5a), the circular symmetry of the waveguiding cores 100 of probing device 30 with respect to the circumference of a lumen 102, ensure equal time of arrival for all the ultrasound reflections. In the latter case (FIG. 5b), reflections are confined to small portions of the lumen circumference as shown in the figure: reflections from only few portions of the circumference (four shown in the figure, indicated by arrows 106) are appreciable, where reflections from other portions of the lumen circumference are directed away from the probe. These two reflections arrive at a different delay to each core. We have already estimated the time difference for two adjacent detecting cores. Here we consider the time difference for the other extreme case where the cores are farthest apart. For example, considering only two cores: core L and core R. The reflection from the right wall arrives first to R and then to L, with a small delay. Conversely, the echo from the left wall arrives first to core L and then to core R. Using the same numerical example as previously: central core diameter of 210 μm with peripheral cores arranged on a diameter of 225 μm, the difference in the arrival time between a signal from the left and a signal from the right is approximately 38 ns. This may not be a significant phase difference at low acoustic frequencies, however, at frequencies typically used in ultrasonic medical imaging, ranging between 10 to 50 MHz, this corresponds to a phase shift of between a third of a period and one and a half periods, respectively. In this example, the phase shifts that are considered are between the peripheral cores that are located farthest apart; other peripheral waveguides also experience such a phase difference that is scaled to their actual physical separation. This phase information should be accounted for when “subtracting waveforms” as is required in the demultiplexing procedures, and can potentially provide important angular information to reconstruct the actual lumen internal profile and wall thickness.

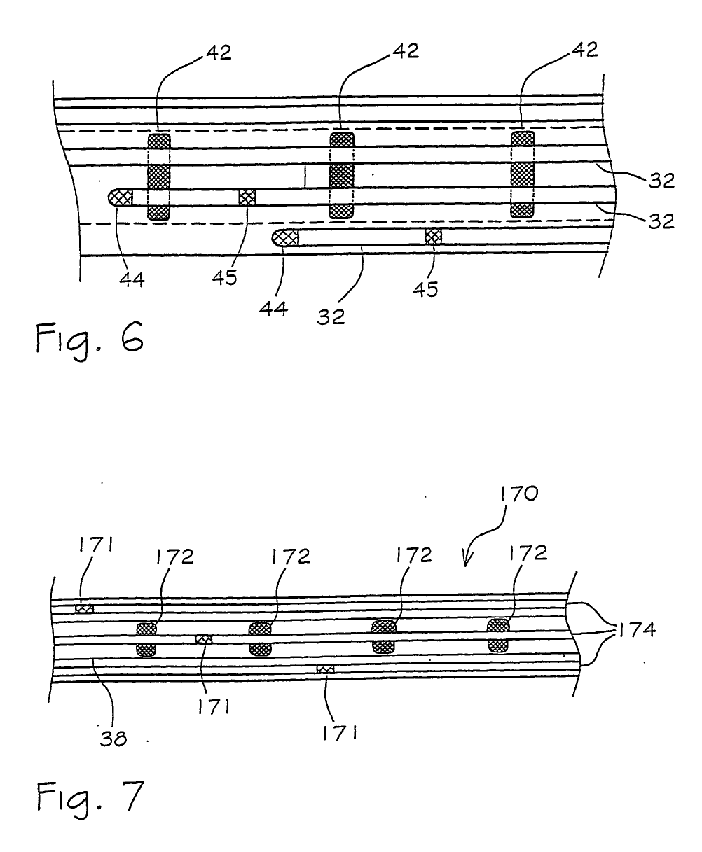

To avoid the effect of time differences between the detecting core and the reference core we consider a modified preferred embodiment in which transmission and detection cores are arranged in a similar manner to the arrangement of FIGS. 3a and 3b, but where, in addition, each detection core, incorporates two detection channels. The channels are configure in a way that the first channel is sensitive to ultrasound throughout its length, whereas the second channel is sensitive throughout its length up to the region of the penultimate ultrasound transmitter. This is shown schematically in FIG. 6, depicting three of the absorbing regions 42, and several detecting cores 32. The detecting cores incorporate here a selective reflector 45, which separates between the two aforementioned optical channels, and a fully reflective element at the end of the core, 44. In such a way the detection channel and the reference channel occupy the same location physically and the difference between the signals detected is confined to the effect of the region at the distal end of the core beyond the selective reflector. This implementation alleviates the difficulty of timing differences between the two channels. The selective reflector can be designed so as to separate the channels on the base of wavelength, polarization or guiding modes. The first option is readily achieved with Bragg gratings that can be induced in the core. The latter two options are implemented with suitable manipulation of the core geometry. An example of such selective reflector 45 is a polarizer or a partial reflecting of the core cross-section.

A further possibility relates to a system with one or more transmitting regions in each core, with the same core sustaining also the detection signals. Here frequency-filtering regions are required to multiplex and demultiplex the detection and generation signals and associate them with different regions in the length of the fiber. Naturally, multiple-application of such multiple detection/transmission fibers can be deployed in one ensemble.

The distribution of the detection cores across the cross-section of the fiber introduces a relative timing delay between the same signal portions arriving at each core. For a basic implementation, which assumes that these errors are sufficiently small to be ignored, these phase differences are model errors. Nevertheless, this situation can be turned around and the phase delays between cores can be used to decode the angular information related to these phase differences. This can provide for a degree of angular resolution of the signal. In principle there can be up to N−1 angular segments for N cores. Similarly, if the sensing regions are made closer together they can be formed into a phase array in the longitudinal direction. In this manner the guidewire can also be made into a two-dimensional imaging system.

Reference is now made to FIG. 7 illustrating a sectional side-view of a probing device in accordance with another preferred embodiment of the present invention having a peripheral waveguide arrangement provided with reflectors, such as Bragg reflectors, whose position is staggered along the length of the fiber. In the embodiment shown in FIG. 3a, the peripheral waveguides are of different length so as to pick-up signals from different portions of the probe, where different absorbing regions are provided. In the embodiment shown in FIG. 7, Bragg grating reflectors are used instead. Fiber 170 is provided with absorbing regions 172 similarly to absorbing regions 42 in probing device 30 (FIG. 3). Independent waveguides 174 are used for detection. Instead of having different lengths, each of these waveguide 174 incorporates a Bragg grating 171 that acts as a reflector and reflects the signal to the proximal end of probing device 170. As mentioned herein above, these waveguides carry demodulated ultrasonic information from which the dimensions of the lumen in which the probe is positioned can be calculated.

Reference is now made to FIG. 8 illustrating a sectional side-view of a probing device in accordance with another preferred embodiment of the present invention having a peripheral waveguide arrangement provided each with one or more selective reflectors, such as Bragg reflectors, whose position is staggered along the length of the fiber. In the embodiment shown in FIG. 7, the peripheral waveguides are used to guide one detecting channel, here two or more detecting channels are incorporated in each fiber. Each such selective reflector determines the extent, and therefore the range of detection of each of the multiplexed cannel. In a way this is an extension of the implementation shown in FIG. 6, whereas the primary intention there is to provide a compensating reference channel to each detecting channel, and here the intention is to provide for multiple independent detection channels. The main advantage of these additional channels is the possibility to form a multi-detector array: each peripheral waveguide can now serve to detect and demultiplex signals received in each region, and the availability of the set of all the peripheral detectors provides for the additional angular information that can expand the applicability of the probing device to the realm of imaging. We note that while Bragg-type reflectors and frequency domain multiplexing is a possible implementation to the selective reflectors in this configuration. Other selective reflector implementations, such as polarization or guided-mode dependent reflectors are also possible to use.

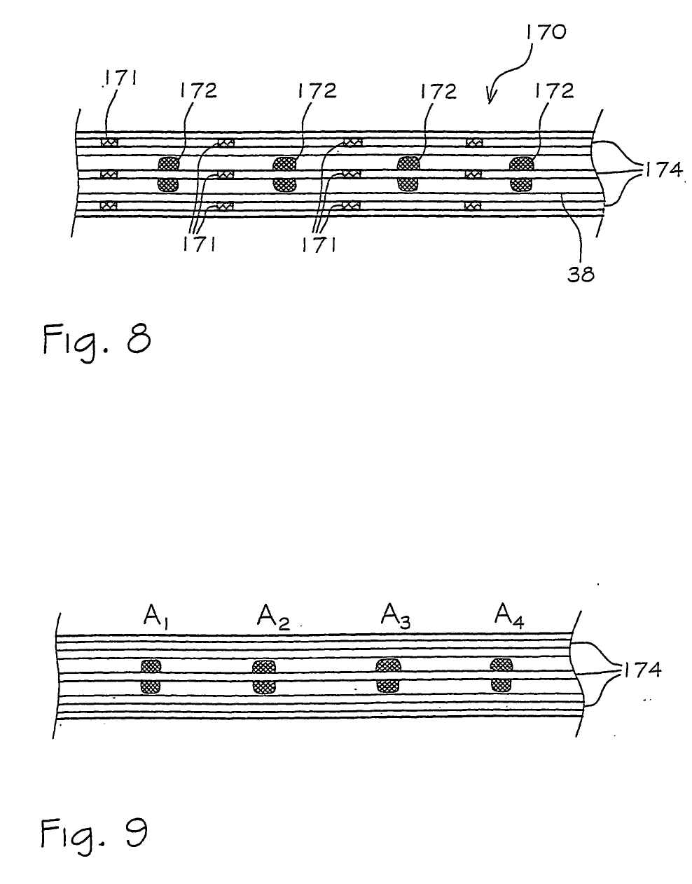

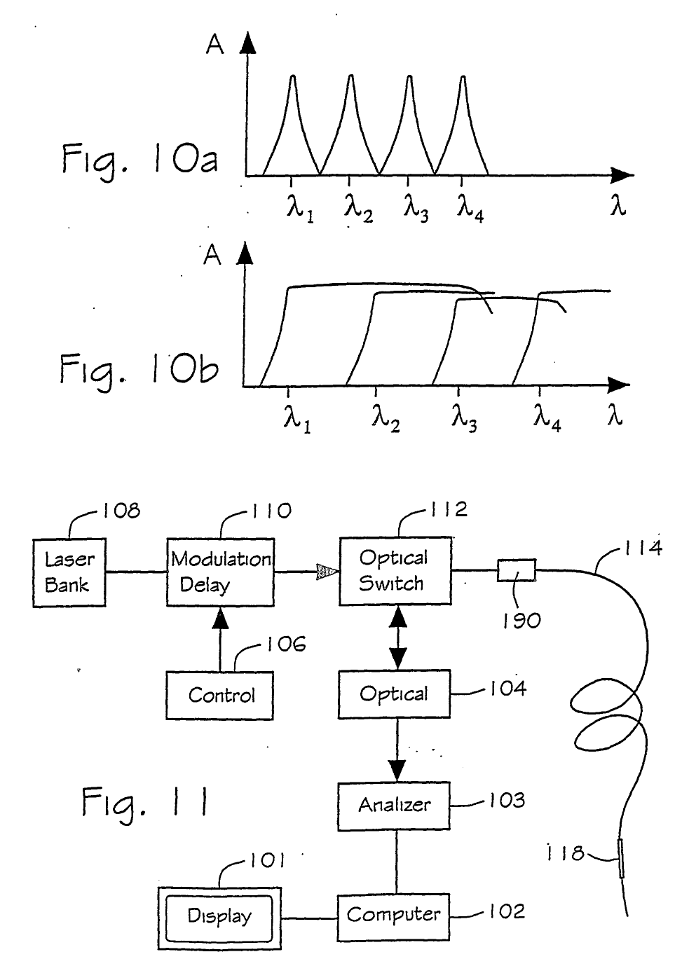

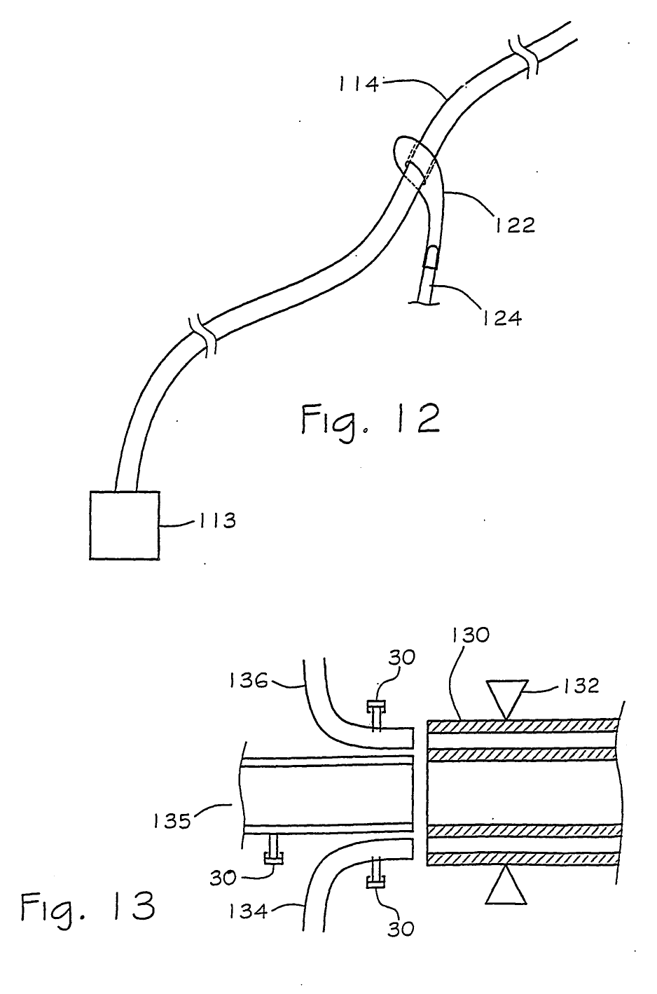

Reference is now made to FIG. 9 illustrating a sectional side-view of a probing device in accordance with yet another preferred embodiment of the present invention having a distribution of selective absorbers that serve to excite ultrasound in different regions according to a predetermined procedure. Here absorbers with different wavelength response are employed. For example, the four absorbing regions shown in FIG. 9 can each be designed to absorb in a limited spectral range, as depicted schematically in FIG. 10a. Here radiation transmitted at wavelength λ₁ is transmitted essentially unperturbed through absorbers A₂, A₃ and A₄, which do not absorb at λ₁ as shown in their spectral response diagrams in FIG. 10a, but shall be absorbed in absorber A₁ to generate ultrasound there. Similarly radiation at λ₂ will be transmitted through absorbers A₃ and A₄ and be absorbed in A₂ to generate ultrasound there only. Interestingly the same effect can be achieved with materials each exhibiting a significantly broader absorption spectrum that may overlap the absorption spectrum of other materials in use; the only requirement is that each wavelength will have an absorption spectrum that extends beyond the absorption spectrum of all the absorbers that precede it. This sort of spectrum is shown in FIG. 10b. As an example for suitable absorbers we refer here to Copper-doped material with absorption spectrum at 450 nm and shorter wavelengths, Alexandrite-doped material with absorption at 850 nm and longer wavelengths, Yitterbium-doped material with absorption in the range 1,000-1300 nm. Absorption in these examples can be made very high such that all the incident radiation is absorbed in a very short distance, on the order of 1 mm. The wavelength division multiplexed generation can be combined with multiple absorbers, for example doubling each of the above absorbing regions such that each wavelength is divided into two absorbers. With such a double region arrangement a total of five different absorbing materials is necessary to construct a ten-element transmitter.

As was discussed in PCT/IL02100018, incorporated herein by reference, the absorber geometry and characteristics can take different forms. In a preferred embodiment of the invention shown herein, different absorbers are provided for different wavelengths. Optionally, each absorber can be designed to absorb several relevant wavelengths. Optionally, there is a spatial overlap between the absorbers for different wavelengths, for example a 0.1 mm region that absorbs a first wavelength includes a 0.05 mm sub-region that absorbs a second wavelength in addition to the first wavelength. Such overlap potentially increases the design flexibility in controlling the acoustic transmission envelope, direction and/or frequency.

Furthermore, the absorption can be volumetric in nature, such that the absorption is gradual along the direction of propagating of the radiation, rather than the energy being absorbed on a surface or boundary layer of the volume. Optionally, the volume is selectively absorbing of wavelength, polarization and/or does not block the entire cross-section of a light guide used to provide the light.

As discussed in PCT/IL02/00018, incorporated herein by reference, a reflector may be provided distal to an absorber, to reflect radiation that is not absorbed by the absorber on the forward pass, back into the same region for further absorption. Optionally, the radiation is made to reverberate several times through the absorber. This may be accomplished, for example, by two reflectors positioned on either side of the absorber. Alternatively a polarization-based two-pass reflecting system can be implemented by providing a polarization-changing element at the distal reflector and/or at the entrance to an absorber (or integrated into the absorber), so that the radiation inside the absorber has a polarization that is reflected by a polarization dependent reflector provided at the entrance to the absorbing volume. Such a polarization dependent reflector may also be provided at the exit from the absorbing volume. Optionally, the reflector(s) and/or the number, size and/or density of the absorbing volume(s) are selected to control the uniformity of the waves generated by one or more regions. A particular region may include absorber density variations along its length and/or cross-section, alternatively or additionally to changes in wavelength-dependent behavior. In a preferred embodiment of the present invention, a plurality of absorbers is included in each of the distributed sensor location and placed along the wave-guide. The type, dimensions and relative positions of these absorbers may be used to determine the characteristics of the generated ultrasound. Suitable arrangements can optionally determine the directionality, spectral contents, waveform, and the intensity of the ultrasonic radiation. A potential benefit of multiple or extended regions is better heat dissipation, possibly allowing higher ultrasonic peak-power to be effectively used.

In a preferred embodiment of the invention, a plurality of absorbers act in concert to provide a desired energy field distribution and/or wave propagation direction. For example, the distance between two absorbing regions may be related to a desired acoustic wavelength to be generated. The absorbing regions that act in concert may be absorbing a same wavelength of radiation or different wavelengths. Alternatively or additionally, the number, spacing and/or length of the regions may be used to select the wavelength spectrum generated in one or more directions. Alternatively or additionally, the regions in the same or different fibers may be used to steer the ultrasonic waves, for example, using phase differences between the regions.

In a preferred embodiment of the present invention, a plurality of absorbers are used to generate a strong acoustic wave while maintaining a low average acoustic radiation power, which radiation power is desirably below a break-down point of the absorbing target The plurality of absorbers allows the target to accumulate a larger overall acoustic power while maintaining the peak power level at each region below a specified threshold.

In a preferred embodiment of the present invention, the ultrasound is generated without any free-space propagation of light, with light going directly from a wave-guide to an absorbing volume. Alternatively, spaces are defined in the waveguide, for example if the waveguide is hollow or by providing air (or vacuum or other fluids or gasses) spaces, such as expansion spaces, adjacent the target.

An aspect of some embodiments of the present invention relates to control ultrasound properties by spatial and density design of absorbing volumes. In a preferred embodiment of the present invention, the control includes one or more uniformity, frequency, number of cycles, directivity and waveform. In another preferred embodiment of the present invention, the control is achieved by providing multiple and suitably spaced absorbing volumes, possibly with different volumes being addressable using different wavelengths, polarizations and/or via different fibers. Alternatively or additionally, the volumes have controlled densities, which may be matched, for example, to the expected relative intensity of an electromagnetic wave at the volume. It should be noted that this control contrasts with that suggested in the art for fluid based systems, in which the absorption depth is fixed and a single volume is used. While the use of solids is desirable in many embodiments of the invention, other material phases, such as gas or liquid may be used. In the example of absorption outside of a catheter, the density of absorbing material may be controlled in order to achieve a desired radiation volume.

An aspect of some embodiments of the present invention relates to providing multiple absorbing regions in a waveguide, for generation of ultrasound from each of the regions.

An aspect of some embodiments of the invention relates to providing multiple electro-magnetic radiation waves in a wave-guide, such that a plurality of functions is provided. The multiple waves may have different polarization and/or wavelengths. In an exemplary embodiment of the invention, one of the waves is used for the generation of ultrasound and another wave is used for detection of ultrasound or treatment based on the radiation. Such treatment may be, for example, treatment using the radiation, treatment using heat or treatment using high-powered ultrasound generated from the radiation. In a preferred embodiment of the invention, ultrasound radiation is generated from the electromagnetic wave during forward traveling of the electro-magnetic wave.

An aspect of some embodiments of the present invention relates to an acousto-optical medical probe that provides a distributed sensing capability over an extended length of the device. The device is incorporated into a mechanical structure such that it can mechanically serve as guide-wire with provisions for independent insertion into an artery and serving as a guide over which, surgical tools can be slide into position. Alternatively, ultrasound detection and/or generation may be by an external probe. In an exemplary embodiment of the invention, the acoustic radiation and light radiation are provided using a same optical fiber.

Reference is made to FIG. 11 illustrating an optical ultrasonic system in accordance with a preferred embodiment of the present invention. An ultrasonic probing device having multiple waveguides 114 comprises at least one optical fiber in a probing device 114 that is similar to one of the probing device configurations considered above. The probing device 114 comprises at least two sensors distributed along an ultrasound generating and/or detecting region 118. Light for generating ultrasound and/or outputting a beam of light at suitable region closer to the distal end 118 is provided by one or more light sources 108 that can preferably be a laser source and/or a flash lamp. In the case of a flash lamp, a filter with one or more spectral pass regions may be provided, for generating a desired spectrum. The light from light sources 108 is optionally modulated (to provide a pulsed source or a different envelope, such as saw-tooth, sinusoidal or one relating to the desired acoustic waveform) by a modulator and delay source 110. The delay or pulsing phase difference between different light beams may be used, for example, to control a beam direction. In some embodiments, the source is self-modulated (e.g., a pulsed laser).

It should be noted that in many embodiments of the present invention, the ultrasonic probing device having distributed sensors may comprise only a single fiber having a relatively small diameter. Optionally, this fiber can be coated with various materials, such as anti-coagulants and biocompatible polymers. Alternatively or additionally, a hollow waveguide can be used.

A coupler or switch 112 is provided for coupling the light to probe 114 and couple detection light from probe 114 to a detector 104. A dedicated processor 103 is provided for data analysis and demultiplexing. A controller 106 controls the generation and detection sequences. Optionally, a computer (e.g., a microcontroller) 102 is provided, with a suitable display 101, for a user interface and/or for storing recorded signals, images and other data.

The multi-core or multi-waveguide characteristics of the distributed ultrasonic probing device, requires a specialized connector to couple probe 190 with the other components and in order to separate the radiation for the generation of ultrasonic signals and the detection of the acoustic signals. In principle, a standard multi-core arrangement can be used, where a mechanical connector is designed to register the angular direction so as to ensure the matching of different cores within the fiber on both sides of the connector. This approach suffers two drawbacks: a) multi-core connectors typically incorporate an inherent relatively large misalignment of the cores; and b) introducing a connector to the end of the device limits its use as a mechanical guide wire: any mechanical connector is significantly larger than the diameters allowed for standard guide wires, substantially 0.34 mm.

Reference is now made to FIG. 12 illustrating a proposed connection between a distributed ultrasonic probing device, light source and detectors in accordance with a preferred embodiment of the present invention. This embodiment represents a first approach in which the connection of the probe is implemented for replacement only. In such an arrangement, standard multi-core fiber connectors can be deployed. This approach is limited with respect to the guiding ability of the probe, and therefore, limited to situations where the surgical device can be guided in along-side the probe rather then over the probe. Probe 114 is hooked up to the rest of the system with a multi-core fiber connector 113. Probe 114 is delivered pre-mounted with a sliding glider 122 that incorporates a mechanical means 124, such as a bayonet hook, for securing the tip of the surgical tools to be guided along the guidewire. The system can now be inserted into position, and then various surgical tools connected to glider 122 may be inserted along the guide wire (not shown in the Figures), and removed for replacement with other tools.

Reference is now made to FIG. 13 illustrating a sectional side view of an alternative connector in accordance with another preferred embodiment of the present invention. The connection incorporates a dynamically-aligning coupler in which a fiber 130 is inserted into an aligning clamp 132 along with a set of individual fibers 134, 135 and 136. The individual fiber position is then perturbed using miniature actuators 30 such as piezoelectric actuators or MEMS. Feedback to the correct position of each fiber is obtained by monitoring the reflected intensity and polarization from the interface of each fiber, monitoring the scattering light off the side of the fibers, and monitoring the visual position of the free fibers and each core. In this manner each fiber is adjusted for best coupling to one of the cores of the multi-core fiber. This arrangement maintains the end of the fiber at its original width, which is sufficiently small by design for performing the mechanical task of guiding surgical tools down the artery.

Reference is now made to FIG. 14 illustrating a sectional side view of a coupler for coupling the probe and other elements in accordance with yet another embodiment of the present invention. In this alternative solution for a coupler, coupler 150 comprises a special preparation of the fiber such that the ends of the individual detecting cores 152 are exposed of the cladding and free to flex. Detecting cores 152 are guided mechanically into position. A suitable cover is included with the device to protect the ends when not in use.

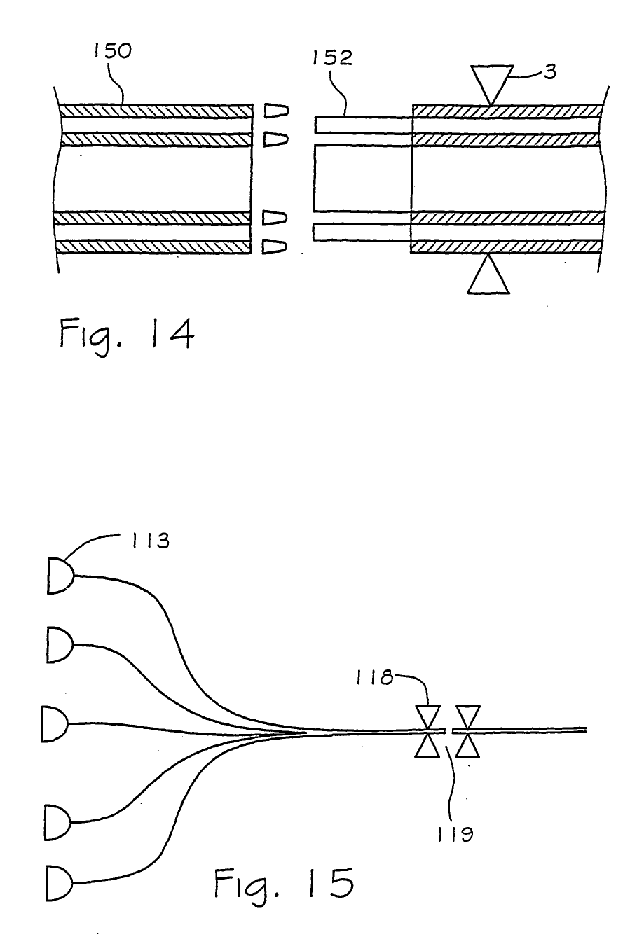

A fourth alternative overcomes the inherent uncertainty of the internal alignment of the different fiber cores. It is this uncertainty in the actual position of the internal cores that make it necessary for the alignment procedures of each core described in the previous two alternatives. To overcome the manufacturing tolerance issues it is proposed to include two connecting systems as shown in FIG. 15. The entire guide-wire assembly is disconnected from the system for replacement using large form-factor connectors, 113. At a suitable distance from the connector the fiber is precut to form an intersection 119. At this location the fiber is mounted into an aligning jig 118. This approach ensures that the cores in the intersection 119, which are cut from the same location in the fiber, are spatially distributed in accurate alignment. The alignment jig must ensure the lateral alignment, the longitudinal spacing the angular tilt and the rotary alignment, comprising six degrees of alignment. Of these it is feasible that the tilt and lateral alignment be affected by mechanical means and the system must dynamically control only the relative fiber separation and relative rotation of the two components. Even if additional degrees of freedom are necessary this approach is still significantly simpler to implement than the former two which require many more dynamic alignment, or mechanical registration.

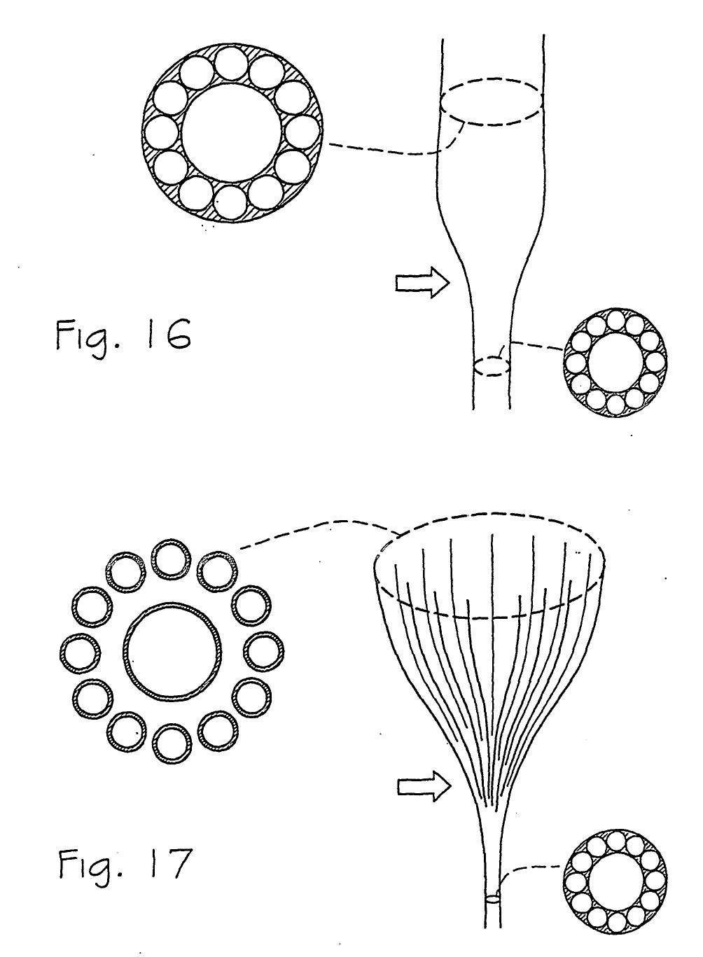

Multicore fibers are available commercially, but mainly for situations where the satellite cores are used for incoherent pumping operation. Here attention is necessary to implement highly accurate connectors for the satellite cores, which need to carry interferometric signals. Two different approaches are introduced to the manufacture of the multicore fiber for the use as a distributed probing device: a) prepare a multicore perform which can then be pulled to effect a multicore fiber (FIG. 16), or b) assemble a bundle of individual fibers which can then be fused together (FIG. 17). The latter procedure can also use over-sized fibers to form the bundle, and the assembly can then be fused and pulled to reduced its dimensions simultaneously. The convenience of manufacturing of option a) above is compromised by the added complexity of effecting a high-quality connector. As the satellite cores carry interferometric signals, and the central core carries a high-power pulse, the connectors must effect an accurate alignment of the cores to minimize signal disruption and loss. The option of using a bundled fiber assembly, while retaining the ends of the fibers free, offers the ability to use individual connectors, or connector arrays, on each fiber in the bundle; the operational implications are certainly unfavorable here; but one can always assure the quality of the coupling in the connector. This option can alternatively use larger fibers that are bundled together, then the bundle fused and pulled to reduce the overall diameter of the assembly. The latter approach is favorable in allowing a significantly more compact fill factor in the fiber cross-section, which is also important in considering the losses to traversing ultrasound.

It is noted that some of the embodiments above described may describe a best mode contemplated by the inventors and therefore include structure, acts or details of structures and acts that may not be essential to the invention and which are described as examples. Structure and acts described herein are replaceable by equivalents, which perform the same function, even if the structure or acts are different, as known in the art. Therefore, the scope of the invention is limited only by the elements and limitations as used in the claims. When used in the following claims, the terms “comprise”, “include”, “have” and their conjugates mean: “including but not limited to”.

1. A probing device for insertion into a duct having a physical structure to determine local parameters associated with the physical structure of the duct at a selected region of the duct, and in particular variations in the physical structure along a predetermined length of interest, the probing device comprising:
at least one of a plurality of waveguides incorporated in an elongated assembly designed to be inserted into the duct;
at least one of a plurality of transmitters, spaced and distributed along a predetermined length of said at least one of a plurality of waveguides incorporated in the elongated assembly, each capable of independently transmitting an acoustic signal of predetermined characteristics;
a plurality of receivers, spaced and distributed along a predetermined length of said at least one of a plurality of waveguides incorporated in the elongated assembly, each capable of receiving echoes of the acoustic signal, reflected off the structure of the duct;
whereby when each of said at least one of a plurality of transmitters generates an acoustic signal, echoes of the signal are received by the plurality of receivers and received data associated with the echoes is processed by a processing unit to determine parameters of the physical structure at the region.

2. The device as claimed in claim 1, wherein at least some of said at least one of a plurality of transmitters and said plurality of receivers are combined in the form of receiving and transmitting transducers.

3. The device as claimed in claim 2, wherein at least some of the transducers are piezo-electric transducers.

4. The device as claimed in claim 1, wherein each of said at least one of a plurality of transmitters, comprises an absorbing region within an optical fiber, the absorbing region made from material, which converts optical signals to acoustic signals.

5. The device as claimed in claim 4, wherein each of said at least one of a plurality of transmitters, comprises at least one of a plurality of absorbing regions within an optical fiber, the absorbing regions made from material, which converts optical signals to acoustic signals.

6. The device as claimed in claim 5, wherein the absorbing regions are made of material that absorbs at different optical spectra, such that at least one of the absorbing regions are activated to generate acoustic signals at a certain optical spectrum, and other absorbing regions are activated to generate acoustic signals at one or more different optical spectra.

7. The device as claimed in claim 5, wherein the absorbing regions are made of material selected from the group containing: Copper-doped material with absorption spectrum at about 450 nm or shorter wavelengths, Alexandrite-doped material with absorption at about 850 nm or longer wavelengths, and Yitterbium-doped material with absorption in the range 1,000-1300 nm.

8. The device as claimed in claim 4, wherein each of said plurality of receivers comprises at least one of a plurality of optical fibers through which light can traverse and be modulated by the echoes.

9. The device as claimed in claim 8, wherein each one of said fibers, serving as a receiver, includes a reflecting element that effectively limits the extents of the fiber.

10. The device as claimed in claim 9, wherein the reflecting element comprises a Bragg grating optical element.

11. The device as claimed in claim 8, wherein at least some of said fibers serving as receivers are staggered along the length of interest to sensitize them to different regions along the length of interest.

12. The device as claimed in claim 11, wherein signals are processed by subtracting signals of two detecting fibers, such that the resulting signal corresponds to their region where the two fibers do not overlap.

13. The device as claimed in claim 8, wherein said fibers, serving as receivers, each include two reflecting elements and two types of light propagating in the fiber effectively forming two detection channels; the distal reflecting element serves to effectively limit the extent of the fiber for one of the detecting channels, and the proximal reflecting element serves to effectively limit the extent of the fiber for the other detecting channel; the differential signal between these two channels effects a sensitive region local to the separation of the two reflecting elements.

14. The device as claimed in claim 13, wherein at least some of said sensitive local regions are staggered along the length of interest to sensitize them to different regions along the length of interest.

15. The device as claimed in claim 13, wherein received signals are processed to account for different phases in each receiver in conjunction with a knowledge of physical separation between the receivers so as to effect a circumferential resolution in the device.

16. The device as claimed in claim 13, wherein at least one of the two reflecting elements comprises a Bragg grating optical element, and the two channels are differentiated by wavelength.

17. The device as claimed in claim 13, wherein at least one of the two reflecting elements comprises a polarization-dependent reflector, and the two channels are differentiated by polarization.

18. The device as claimed in claim 13, wherein at least one of the two reflecting elements comprises a spatially selective element, reflecting one or more guided modes, and the two channels are differentiated by guided modes.

19. The device as claimed in claim 4, wherein each of said plurality of receivers comprises at least one of a plurality of optical fibers through which light can traverse and be modulated by the echoes and which incorporates several wavelength-dependent reflectors, such that each effectively limits extent of a certain optical wavelength traveling in the fiber; the position of at least some of these reflecting elements is distributed along the length of the interest, sensitizing each wavelength to a different positions along the length of interest.

20. The device as claimed in claim 19, wherein the received signals are processed to account for the different phases in each receiver in conjunction with a knowledge of physical separation between the receivers so as to effect a circumferential resolution in the device.

21. The device as claimed in claim 1, wherein each of said at least one of a plurality of transmitters, comprises at least one absorbing region within a multicore optical fiber, the absorbing region made from material, which converts optical signals to acoustic signals, and wherein at least one of the cores serve as at least one receiver.

22. The device as claimed in claim 21, wherein some of the cores serving to generate the acoustic signals have larger lateral cross section than those serving for detection.

23. The device as claimed in claim 21, wherein the cores in the said multicore optical fiber, serving as receivers, include a reflecting element that effectively limits the extent of each of the receiver cores and sensitizes each on to a different positions along the length of interest.

24. The device as claimed in claim 23, wherein the reflecting element comprises a Bragg grating optical element.

25. The device as claimed in claim 21, wherein said cores, serving as receivers, include two reflecting elements and two types of light propagating in each core effectively forming two detection channels; the distal reflecting element serves to effectively limit the extents of the core for one of the detecting channel, and the proximal reflecting element serves to effectively limit the extent of the core for the other channel; the differential signal between these two channels effects a sensitive region local to the separation of the two reflecting elements.

26. The device as claimed in claim 25, wherein at least one of the two is reflecting elements comprises a Bragg grating optical element, and the two channels are differentiated by wavelength.

27. The device as claimed in claim 25, wherein at least one of the two reflecting elements comprises a polarization-dependent reflector, and the two channels are differentiated by polarization.

28. The device as claimed in claim 25, wherein at least one of the two reflecting elements comprises a spatially selective element, selectively reflecting one or more guided modes, but not reflecting other such modes, and the two channels are differentiated by different guided modes.

29. The device as claimed in claim 1, wherein said predetermined length of the elongated structure extends to approximately 30 mm.

30. A probing device for insertion into a duct having a physical structure to determine local parameters associated with the physical structure of the duct at a selected region of the duct, and in particular variations in the physical structure along a predetermined length of interest, the probing device comprising:
an elongated assembly designed to be inserted into the duct;
a plurality of transmitters, spaced and distributed along a predetermined length of said elongated assembly, each capable of independently transmitting an acoustic signal of predetermined characteristics;
at least one of a plurality of receivers, spaced and distributed along a predetermined length of said elongated assembly, each capable of receiving echoes of the acoustic signal, reflected off the structure of the duct;
whereby when each of said plurality of transmitters generates an acoustic signal, echoes of the signal are received by the at least one of a plurality of receivers and received data associated with the echoes is processed by a processing unit to determine parameters of the physical structure at the region.

31. The device as claimed in claim 30, wherein said plurality of transmitters and said at least one of a plurality of receivers are combined in the form of receiving and transmitting transducers.

32. The device as claimed in claim 31, wherein at least some of the transducers are piezoelectric transducers.

33. The device as claimed in claim 30, wherein each of said plurality of transmitters, comprises an absorbing region within an optical fiber, the absorbing region made from material, which converts optical signals to acoustic signals.

34. The device as claimed in claim 33, wherein each of said plurality of transmitters, comprises at least one of a plurality of absorbing regions within an optical fiber, the absorbing regions made from material, which converts optical signals to acoustic signals.

35. The device as claimed in claim 34, wherein the absorbing regions are made of material that absorbs at different optical spectra, such that at least one of the absorbing regions are activated to generate acoustic signals at a certain optical spectrum, and other absorbing regions are activated to generate acoustic signals at one or more different optical spectra.

36. The device as claimed in claim 34, wherein the absorbing regions are made of material selected from the group containing: Copper-doped material with absorption spectrum at about 450 nm or shorter wavelengths, Alexandrite-doped material with absorption at about 850 nm or longer wavelengths, and Yitterbium-doped material with absorption in the range 1,000-1300 nm.

37. The device as claimed in claim 33, wherein each of said at least one of a plurality of receivers comprises at least one of a plurality of optical fibers through which light can traverse and be modulated by the echoes.

38. The device as claimed in claim 37, wherein said fibers, serving as receivers, each include two reflecting elements and two types of light propagating in the fiber effectively forming two detection channels; the distal reflecting element serves to effectively limit the extent of the fiber for one of the detecting channels, and the proximal reflecting element serves to effectively limit the extent of the fiber for the other detecting channel; the differential signal between these two channels effects a sensitive region local to the separation of the two reflecting elements.

39. The device as claimed in claim 38, wherein each of said fibers serving as receivers are staggered along the length of interset to sensitize them to different regions along the device.

40. The device as claimed in claim 38, wherein the received signals are processed to account for the different phases in each receiver in conjunction with a knowledge of their physical separation so as to effect a circumferential resolution in the device.

41. The device as claimed in claim 38, wherein at least one of the two reflecting elements comprises a Bragg grating optical element, and the two channels are differentiated by wavelength.

42. The device as claimed in claim 38, wherein at least one of the two reflecting elements comprises a polarization-dependent reflector, and the two channels are differentiated by polarization.

43. The device as claimed in claim 38, wherein at least one of the two reflecting elements comprises a spatially selective element, selectively reflecting one or more guided modes, and the two channels are differentiated by guided modes.

44. The device as claimed in claim 37, wherein each one of said fibers, serving as a receiver, includes a reflecting element that effectively limits the extents of the fiber.

45. The device as claimed in claim 44, wherein the reflecting element comprises a Bragg grating optical element.

46. The device as claimed in claim 44, wherein each of said fibers serving as receivers are staggered in their length to sensitize them to different regions along the length of interest.

47. The device as claimed in claim 46, wherein signals are processed by subtracting signals of two adjacent detecting fibers, such that the resulting signal corresponds to their region where the two fibers do not overlap.

48. The device as claimed in claim 33, wherein each of said plurality of receivers comprises an optical fiber through which light can traverse and be modulated by the echoes and which incorporates several wavelength-dependent reflectors, such that each effectively limits extent of a certain optical wavelength traveling in the fiber; the position of these reflecting elements is distributed along the predetermined length of the device, sensitizing each wavelength to a different positions along the assembly.

49. The device as claimed in claim 48, wherein the received signals are processed to account for the different phases in each receiver in conjunction with a knowledge of their physical separation so as to effect a circumferential resolution in the device.

50. The device as claimed in claim 30, wherein each of said at least one of a plurality of transmitters, comprises at least one absorbing region within a multicore optical fiber, the absorbing region made from material, which converts optical signals to acoustic signals, and wherein several of the cores serve as one or more receivers.

51. The device as claimed in claim 50, wherein some of the cores serving to generate the acoustic signals have larger lateral cross section than those serving for detection.

52. The device as claimed in claim 50, wherein the cores in the said multicore optical fiber, serving as receivers, include a reflecting element that effectively limits the extent of each of the receiver cores and sensitizes each on to a different positions along the assembly.

53. The device as claimed in claim 52, wherein the reflecting element comprises a Bragg grating optical element.

54. The device as claimed in claim 50, wherein said fiber, serving as receiver, includes two reflecting elements and two types of light propagating in the fiber effectively forming two detection channels; the distal reflecting element serves to effectively limit the extents of the fiber for one of the detecting channel, and the proximal reflecting element serves to effectively limit the extent of the fiber for the other channel; the differential signal between these two channels effects a sensitive region local to the separation of the two reflecting elements.

55. The device as claimed in claim 54, wherein at least one of the two reflecting elements comprises a Bragg grating optical element, and the two channels are differentiated by wavelength

56. The device as claimed in claim 54, wherein at least one of the two reflecting elements comprises a polarization-dependent reflector, and the two channels are differentiated by polarization.

57. The device as claimed in claim 54, wherein at least one of the two reflecting elements comprises a spatially selective element, reflecting one or more guided modes, and the two channels are differentiated by guided modes.

58. The device as claimed in claim 30, wherein said predetermined length of the elongated structure extends to approximately 30 mm.

59. A system for determining local parameters associated with a physical structure of a duct at a selected region of the duct, and in particular their variation of a predetermined length of interest, the system comprising:
at least one of a plurality of waveguiding structures incorporated with an elongated assembly designed to be inserted into the duct;
a plurality of transmitters, spaced and distributed along a predetermined length of said at least one of the plurality of waveguides incorporated with the elongated assembly, each capable of transmitting an acoustic signal of predetermined characteristics;
at least one of a plurality of receivers, spaced and distributed along a predetermined length of said at least one of the plurality of waveguides incorporated with the elongated assembly, each capable of receiving echoes of the acoustic signal, reflected off the structure of the duct;
a processing unit for processing the received echoes;
a controller for actuating and controlling the operation of the generator and the processing unit,
whereby when each of said at least one of a plurality of transmitters generates an acoustic signal, echoes of the signal are received by at least one of the plurality of receivers and received data associated with the echoes is processed by a processing unit to determine parameters of the physical structure at the region.

60. A method for determining local parameters associated with a physical structure of a duct at a selected region of the duct, and in particular variations in the physical structure along a predetermined length of interest, the method comprising:
providing a system comprising:
a probing device comprising at least one of a plurality of waveguiding structures incorporated within an elongated assembly designed to be inserted into the duct; at least one of a plurality of transmitters, spaced and distributed along a predetermined length of said at least one of the plurality of waveguiding structures incorporated with the elongated assembly, each capable of transmitting an acoustic signal of predetermined characteristics; and at least one of a plurality of receivers, spaced and distributed along a predetermined length of said at least one of the plurality of waveguides incorporated with the elongated structure, each capable of receiving echoes of the acoustic signal, reflected off the structure of the duct;
a processing unit for processing the received echoes;
a controller for actuating and controlling the operation of the generator and the processing unit,
inserting the probing device within the duct at a desired target;
generating an acoustic signal by each of said at least one of a plurality of transmitters;
receiving echoes of the acoustic signal;
processing data associated with the echoes by the processing unit to determine parameters associated with a physical structure of a duct at the desired region.
